(19) United States
(12) Patent Application Publication
(10) Pub. No.: US 2025/0367240 A1
(43) Pub. Date: Dec. 4, 2025

(54) DERIVATION OF GLUCOSE-RESPONSIVE INSULIN-SECRETING CELLS AND ORGANOIDS FROM HUMAN STOMACH CELLS AND THEIR USE TO TREAT DIABETES

(71) Applicant: CORNELL UNIVERSITY, Ithaca, NY (US)

(72) Inventors: Qiao Joe ZHOU, Ithaca, NY (US); Xiaofeng HUANG, Ithaca, NY (US)

(73) Assignee: CORNELL UNIVERSITY, Ithaca, NY (US)

(21) Appl. No.: 18/870,864

(22) PCT Filed: Jun. 2, 2023
(86) PCT No.: PCT/US2023/024294

§ 371 Date: Dec. 2, 2024

Related U.S. Application Data
(60) Provisional application No. 63/348,610, filed on Jun. 3, 2022.

(51) Int. Cl. A61K 35/39; A61K 38/28; A61P 3/10; C12N 5/071

(52) U.S. Cl. CPC A61K 35/39; A61K 38/28; A61P 3/10; C12N 5/0679; C12N 2501/999

(57) ABSTRACT

The present disclosure is directed to a method of producing human gastric insulin-secreting (GINS) cells and human gastric insulin-secreting (GINS) organoids. The present disclosure provides a population of human gastric insulin-secreting (GINS) cells and a preparation of human gastric insulin-secreting (GINS) organoids, which are glucose-responsive and insulin-secreting. The present disclosure also provides methods of controlling glycemia in a human subject by transplanting to the human subject the population of human GINS cells or the preparation of human GINS organoids.

### CROSS REFERENCE TO RELATED APPLICATION

This application claims the benefit of priority from U.S. Provisional Application No. 63/348,610, filed on Jun. 3, 2022, the entire content of which is incorporated herein by reference.

### BACKGROUND

Diabetes Mellitus has reached epidemic levels around the world, with over 11% of U.S. population diabetic. While many drugs are available to manage diabetes, insulin remains the only tool to manage severe hyperglycemia in type 1 diabetes (T1D, ˜1.6 million patients in U.S) and advanced type 2 diabetes (T2D, ˜7.4 million T2D insulin users in U.S). Daily insulin injections, however, incur significant physical and emotional burdens on the patients. Moreover, insulin injections often fail to consistently control blood glucose levels within the normal range. As a result, insulin-dependent patients may develop long-term complications such as retinopathy and neuropathy, with the lifespan of T1D patients shortened by as much as 10 years.

Pancreatic β-cells are the only cell type in the body that makes insulin. β-cell destruction or dysfunction leads to T1D or T2D, respectively. Cadaveric islet transplantation has been practiced for over 20 years and shown to be an effective therapy to control glycemia. However, few cadaveric donors are available, severely limiting its wide therapeutic use.

Gut stem cells are highly proliferative and power the weekly self-renewal of the gut mucosal lining (Gehart et.al., 2019, Nat Rev Gastroenterol Hepatol 16, 19-34; Wells et.al., 2014, Development 141, 752-760; Santos et.al., 2018, Trends Cell Biol 28, 1062-1078). Harvested from biopsies, human gut stem cells can be propagated in culture as organoids or primary cell lines over many generations, providing abundant tissues for potential autologous transplantation therapies (Sugimoto, et.al., 2021, Nature 592, 99-104; Nikolaev et.al., 2020, Nature 585, 574-578; Meran et.al., 2020, Nat Med 26, 1593-1601). Gut stem cells produce gut-specific tissues, including hormone-secreting enteroendocrine cells (EECs). Rare insulin expressing EECs have been reported in fetal human small intestine (Egozi et.al., 2021, Nat Med 27, 2104-2107). Whether such cells secret insulin is unknown, but their presence suggests an intrinsic permissiveness for insulin production in the fetal if not postnatal intestine. Prior to this discovery, it was shown that suppressing Fox01 could activate insulin in murine intestinal EECs (Talchai, et.al, 2012, Nat Genet 44, 406-412) and that pancreatic endocrine-like cells could be generated from putative human endocrine progenitors (Wang et.al., 2015, Nature 522, 173-178). It was also reported that co-expression of the endocrine regulator NEUROG3 (also known as NGN3) and pancreatic β-cell regulators PDX1 and MAFA could induce insulin-secreting cells from murine intestine and stomach (Ariyachet, et.al, 2016, Cell Stem Cell 18, 410-421; Chen et.al., 2014, Cell Rep 6, 1046-1058). However, the same approaches yielded few insulin producers from human gut organoids^{11,12} (Chen et.al., 2014, Cell Rep 6, 1046-1058; Bouchi et.al., 2014, Nat Commun 5, 4242).

Generating functional insulin-secreting cells has tremendous therapeutic value, offering treatments for insulin-dependent diabetes, including the autoimmune type 1 diabetes (Warshauer et.al., 2020, Cell Metab 31, 46-61; Zhou et. al., 2018, Nature 557, 351-358; Brusko et.al., 2021, Science 373, 516-522; Ramzy et.al., 2021, Cell Stem Cell 28, 2047-2061; Sneddon et.al., 2018, Cell Stem Cell 22, 810-823; Millman et.al, 2017, Diabetes 66, 1111-1120). An attractive feature of using gut stem cells to make 3-cell mimics is the ease of establishing autologous organoids from biopsies, which can enable mass production and personalized therapies. Aside from the current technical inability to differentiate gut stem cells into functional β-like cells at sufficient efficiency, a significant unknown factor is the documented short lifespans of gut cells in vivo, numbering in days to several weeks (Barker et.al., 2010, Cell Stem Cell 6, 25-36; Barker et.al., 2007, Nature 449, 1003-1007). This raises the concern as to whether insulin-secreting cells made from human gut tissues will be sufficiently stable and durable as an engraftable therapeutic.

### SUMMARY OF THE DISCLOSURE

To overcome the bottleneck of islet supplies, a novel methodology has been developed to produce glucose-responsive and insulin-secreting cells from cultured human gastric tissues, which can be further aggregated into islet-like organoids. The induction is achieved by expression of three factors (NGN3, PDX1 and MAFA, or “NPM” factors). In some embodiments, the induction is achieved by transient expression of NGN3, followed by stable expression of PDX1 and MAFA. The organoids prepared herein contain predominantly (e.g., about 70%) Gastric INsulin-Secreting cells (GINS cells) that closely resemble pancreatic β-cells in molecular signatures. The organoids also contain other endocrine cells that express one or multiple of the hormones including glucagon, somatostatin, and Ghrelin.

One aspect of the present disclosure is directed to a method of producing human gastric insulin-secreting (GINS) cells comprising: obtaining and culturing gastric stem and progenitor cells from a gastric tissue sample of a human subject; manipulating the gastric stem and progenitor cells to cause the gastric stem and progenitor cells to express a NGN3 factor, followed by a PDX1 factor, and a MAFA factor; and culturing the manipulated cells in a serum free medium to obtain the human GINS cells, wherein the human GINS cells are insulin-secreting and glucose-responsive.

In some embodiments, the factors are exogenously introduced into the gastric stem and progenitor cells. In some embodiments, the factors are induced endogenously by treatment with one or more chemical compounds.

In some embodiments, the factors are exogenously introduced into the gastric stem and progenitor cells by transduction of a viral vector, mRNA transduction, genetic engineering, or a combination thereof.

In some embodiments, the viral vector is a lentiviral vector or an AAV vector. In some embodiments, the genetic engineering method uses CRISPR or TALEN.

In some embodiments, the NGN3 factor is expressed for at least 1 day. In some embodiments, the NGN3 factor is expressed for 2 days.

In some embodiments, the PDX1 factor and the MAFA factor are stably expressed. In some embodiments, the expression of the NGN3 factor is transient, followed by stable expression of the PDX1 factor and the MAFA factor. In some embodiments, the expression of the NGN3 factor lasts for 1-3 days (e.g., 2 days), followed by stable expression of the PDX1 factor and the MAFA factor for at least 2 to 6 days. In some embodiments, the expression of the NGN3 factor lasts for 2 days. In some embodiments, the stable expression of the PDX1 factor and the MAFA factor last for at least 2-4 days.

Another aspect of the disclosure is directed to a method of producing human gastric insulin-secreting (GINS) organoids comprising culturing the human GINS cells in a GINS medium for a period of time to allow aggregation of the human GINS cells into human GINS organoids, wherein the human GINS organoids are pancreatic islet-like organoids, insulin-secreting and glucose-responsive.

In some embodiments, the period of time is from about 6 days to about 21 days. In some embodiments, the period of time is about 10 days.

In some embodiments, the GINS medium is a chemically defined, serum free medium. In some embodiments, the chemically defined, serum free GINS medium comprises N2, B27, and N-acetyl cysteine (“NAC”) in a basal medium.

In some embodiments, the basal medium is supplemented with HEPES, GlutaMAX, Primocin, NAC, B-27, N-2, Nicotinamide, A8301, and Y-27632. In some embodiments, the basal medium is supplemented with 10 mM HEPES, 1× GlutaMAX, 25 μM Primocin, 500 μM NAC, 1× B-27, 1× N-2, 10 mM Nicotinamide, 1 μM A8301, and 10 μM Y-27632.

Another aspect of the disclosure is directed to a population of human gastric insulin-secreting (GINS) cells. In some embodiments, a population of human gastric insulin-secreting (GINS) cells, wherein the human GINS cells: (a) are glucose-responsive and insulin-secreting, (b) do not express certain β-cell markers such as NKX6-1 and GAD65, (c) secrete insulin but having a granule morphology different from that of islet β-cells, and (d) retain residual gastric gene expression.

In some embodiments, the residual gastric gene expression is determined by single cell RNA sequencing. In some embodiments, the granule morphology of the secreted insulin is determined by electron microscopy.

In some embodiments, the human GINS cells express human β-cell markers G6PC2, GCK, ABCC8, NKX2-2, PCSK1 and PAX6. In some embodiments, the human GINS cells do not express human β-cell marker NKX6-1.

Another aspect of the disclosure is directed to a preparation of human gastric insulin-secreting (GINS) organoids, wherein the human GINS organoids comprise human GINS cells that: (a) are glucose-responsive and insulin-secreting, (b) do not express β-cell markers such as NKX6-1 and GAD65, (c) secrete insulin but having a granule morphology different from that of islet β-cells, and (d) retain residual gastric gene expression.

In some embodiments, a method of controlling glycemia in a human subject, comprising transplanting to the human subject the population of human GINS cells or the preparation of human GINS organoids.

In some embodiments, the population of human GINS cells or the preparation of human GINS organoids are transplanted in the liver, muscle(s), a subcutaneous space, a fat depot, an omentum membrane, or an abdominal cavity of the human subject.

In some embodiments, the population of human GINS cells or the preparation of human GINS organoids are autologous or allogenic relative to the human subject.

In some embodiments, the human subject is a human subject having type 1 diabetes, type 2 diabetes, or having a partial or complete pancreatectomy.

In some embodiments, the population of human GINS cells or the preparation of human GINS organoids are mixed, prior or during transplantation, with other cells including mesenchymal cells, vascular cells, or immune cells. In some embodiments, the population of human GINS cells or the preparation of human GINS organoids are mixed, prior or during transplantation, with compounds, growth factors, mRNA, other chemical, protein, and bio or synthetic materials. In some embodiments, the population of human GINS cells or the preparation of human GINS organoids are encapsulated or seeded into a device prior or during transplantation.

Another aspect of the disclosure is directed to a method of treating diabetes in a human subject comprising transplanting to the human subject a mixture of the population of human GINS cells and the preparation of human GINS organoids.

### BRIEF DESCRIPTION OF THE DRAWINGS

The file of this patent or application contains at least one drawing executed in color. Copies of this patent or application with color drawing(s) will be provided by the Patent and Trademark Office upon request and payment of the necessary fee.

FIGS. 1a-f. Generation of Gastric Insulin-Secreting (GINS) organoids from human stomach samples. a, Diagram showing key steps of GINS organoid generation. Medium supplements for hGSC (human gastric stem cell) culture or GINS organoid induction are indicated, with up and down arrows indicating agonists or antagonists, respectively. b, Representative images of human stomach samples, hGSC colonies, and GINS organoids. 1000 cells per organoids. c, d, Immunofluorescent staining and quantification of day-21 GINS organoids for C-peptide (CPPT), MAFA, glucagon (GCG), somatostatin (SST), and ghrelin (GHRL). For CPPT⁺ cell quantification, n=33 independent organoids (donor #6), for other hormone quantification, n=6 independent organoids (donor #6). e, Quantitative flow cytometry of day-21 GINS organoids (donor #10) with negative control. f, Insulin content of day-18 GINS organoids (n=11 different batches of organoids from donor #6) and human islets (n=3 samples from independent donors). Data are mean±s.d.; two-tailed unpaired t-test.

FIGS. 2a-c. GINS organoids secret human insulin in response to glucose and GLP-1 analogue. a, Glucose-stimulated insulin secretion of GINS organoids from donor #6 at different time points (days post differentiation, n=4 independent groups of same batch organoids per time point), from different batches (n=4 (batch1-3) or n=3 (batch 4) independent groups of organoids per batch), or from donor #10 (n=4 independent batches of organoids). For GINS organoids from donor #6, two-way repeated-measures ANOVA with Holm-Sidak's multiple comparisons test. For GINS organoids from donor #10 and islets, one-tailed paired t-test. b, Insulin secretion of day-18 GINS organoids in response to sequential glucose challenges and KCl depolarization (n=5 groups of organoids from donor #6, from two independent experiments). One-way repeated measures ANOVA with Holm-Sidak's multiple comparisons test. c, Dynamic glucose-stimulated insulin secretion by perifusion assay of day-21 GINS organoids derived from donor #6 and #10 (n=4 independent groups of organoids for each donor). a, b, c, Data presented as mean±s.d.

FIGS. 3a-d. Four endocrine cell types identified in GINS organoids by scRNA-seq. a, UMAP visualization of integrated GINS organoid (day 21, n=1, donor #6) and primary human islet single-cell transcriptomes (n=4 independent donors). b, Relative expression of endocrine cell type-specific markers. The shading displays scaled average gene expression, and diameter denotes fractional expression. c, Violin plots of key b-cell function and identity markers in GINS b-like and islet b cells. d, Cell identity scoring with gastric and b-cell signatures (868 and 1,034 genes, respectively). Mucus: mucus-secreting cells spontaneously differentiated from hGSCs in culture; Stem: hGSCs; b-like: GINS b-like cells; islet-b: islet b-cells.

FIGS. 4a-g. Transplanted GINS organoids secrete human insulin and reversed diabetes in mice. a, b, GINS cells (0.8×10⁶, corpus donor #6) were transplanted under the renal capsule of non-diabetic NSG mice, which yielded grafts containing CD31⁺ vascular cells and predominant INS⁺ cells that co-expressed PAX6, MAFA, NKX2-2, PCSK1, and the mature b cell-associated marker ENTPD3 (6 months post transplantation). A small number of SST⁺, GCG⁺, and GHRL⁺ were also found. Repeated independently 3 times with similar results. (a). Human insulin from individual mouse was measured at 7 weeks (cohort-1) and 11 weeks (cohort-2) post transplantation, grafted with different batches of cells, after overnight fasting (blue bar) and 60 minutes after glucose challenge (red bar) (b). c, d, STZ-induced diabetic NSG mice were transplanted with 6-8 million GINS cells from donor #6 (GINS #6, n=5 independent batches of organoids transplanted into 5 individual mice) or 6 million GINS cells from donor #10 (GINS #10, n=3 independent batches of organoids transplanted into 3 independent mice) or without transplantation (Sham). Random-fed blood glucose levels (c) and intraperitoneal glucose tolerance tests (IPGTT, 2 weeks post transplantation) (d) showed significant improvement for both transplanted groups. Data presented as mean±s.e.m.; two-way repeated-measures ANOVA. Tx: transplantation. Nx: nephrectomy. e, 24-hour pulse-chase with EdU revealed no proliferative cells in GINS graft but abundant replicating cells in the intestine of the same engrafted mouse. Repeated independently 3 times with similar results. f, Violin plots of select genes in GINS b-like cells before and after transplantation and comparison with islet b cells. g, Assessment of transcriptomic heterogeneity among GINS b-like cells before and after transplantation and among primary human islet b cells with correlation coefficient analysis across 2,000 top variable genes.

FIGS. 5a-f. Developmental trajectory of GINS cells. a, Experimental design of sampling cells with scRNA-seq at key stages of hGSC differentiation to GINS organoids. UMAP showing clustering of hGSCs (day 0, yellow, n=1, donor #6), endocrine progenitors (day 2, green, n=1, donor #6), GINS precursors (day 6, blue, n=1, donor #6), GINS organoids (day 20, red, n=1, donor #6) and primary human islets (gray, n=1, donor #1). b, UMAP of cell types colored according to their identities. Stem: hGSCs; Endo 1: endocrine progenitors 1; Endo 2: endocrine progenitors 2; GINS pre: GINS precursors. c, Relative expression of select markers across cell types. d, Pseudotime trajectory analysis of cell types. e, Violin plot showing the expression levels of the Pdx1-Mafa transgenes in different cell types. f, Heat map showing gene-expression clusters along the pseudotime trajectory from hGSCs to GINS cells and human islet b cells. Select significant GO terms enriched in each gene cluster are shown. P-value calculated by hypergeometric distribution followed by Benjamini-Hochberg adjustment.

FIGS. 6a-h. Galanin⁺ precursors give rise to GINS cells. a, Relative expression of INS and GAL in GINS precursors, GINS b-like cells and human islet b cells. UMAP subset of FIG. 5b. b, Purification of GFP^{high} GINS precursors differentiated from GAL-GFP hGSC reporter line at day 7 post differentiation. c, d, Quantification of GAL⁺ and CPPT⁺ cell percentage in organoids and their average staining intensity at day 1 or 14 post sorting. Data presented as mean±s.d. (GAL⁺ at day 1: n=9; GAL⁺ at day 14: n=10; CPPT⁺ at day 1: n=13; CPPT⁺ at day 14: n=8; all independent organoids from donor #6); two-way ANOVA with Sidak's multiple comparisons test. e, Representative images of immunofluorescent staining for GAL and CPPT at day 1 and day 14 post sorting. f, Representative immunofluorescence of GAL and INS in primary human islet grafts in NSG mice. Arrows indicate GAL⁺ INS⁺ cells. g, Violin plot showing scRNA levels of GAL in GINS b-like cells before and after transplantation and in human islet b cells. h, Proposed model for GINS organoid formation, with rerouting of the hGSC developmental trajectory by NGN3, PDX1 and MAFA.

FIGS. 7a-e. Optimizing conditions to induce insulin-expressing cells from cultured human gastric stem cells. a, Top: stomach samples from 3 different donors; middle, primary hGSC colonies derived from the stomach samples; bottom, immunofluorescence of passage-10 hGSC colony staining for SOX9 and KI67. b, Growth kinetics of hGSCs from three different donors. c, Co-expression of Ngn3, Pdx1, and Mafa using a polycistronic inducible construct in hGSCs yielded low levels of insulin expression (n=3 biological independent samples). Ubc: ubiquitin promoter. d, To optimize the relative timing of Ngn3 and Pdx1-MafA expression, we expressed a Ngn3ER fusion protein in which Ngn3 activity was induced by 4-OH Tamoxifen (4-OH-TAM). Polycistronic PDX1 and MAFA co-expression was controlled by rtTA-TetO and activated by the addition of Doxycycline in the culture medium. Higher INS expression was achieved by sequential activation of the transcription factors NGN3 and PDX1-MAFA. n=3 independent experiments. e, Comparison of PDX1-MAFA with the other transcription factor combinations in insulin induction. 2-day Ngn3ER induction (by 4-OH-TAM) preceded the other TFs, or alternatively, co-expression cassettes were used. ND: not detected. n=3 independent experiments. c, d, e, Data presented as mean±s.d.; two-tailed unpaired t-test (c), or one-way ANOVA with Dunnett multiple comparisons test (d, e).

FIGS. 8a-e. Formulating chemically defined serum-free medium for GINS organoid differentiation. a, Experimental design for the supplement screen. Ngn3ER: fusion gene in which NGN3 activity was induced by 4-OH-TAM. Polycistronic PDX1 and MAFA co-expression was controlled by rtTA-TetO and activated by the addition of Doxycycline in the culture medium. mCherry was co-expressed with Ngn3ER in the cell line. Ngn3 was activated from day 0 to day 2. Culture medium was switched to a basal serum-free medium (advanced DMEM/F12, 10 mM HEPES, 1× GlutaMAX, 1× B-27, 1× N-2, and 500 μM N-Acetyl-LCysteine) on day 2 with addition of a single supplement and Doxycycline. b, The list of supplements that were screened and the pathways they targeted. Up and down arrows indicate agonists or antagonists, respectively. c, Relative expression of INS mRNA on day-7 post differentiation in comparison with no supplement control. N=3 (treatments) or 5 (control) independent samples. Nicotinamide and Y-27632 treatment significantly up-regulated INS. d, Spontaneous clustering of cells was evaluated by mCherry live imaging on day-7 post differentiation. Select conditions were shown. A8301 treatment had the most observable clustering effect on the nascent GINS cells. Repeated independently 3 times with similar results. e, Relative expression of β-cell markers measured on day-7 post differentiation in comparison with no supplement control. N=6 independent samples. c, e, Data presented as mean±s.d.; one-way ANOVA (c), or two-way ANOVA (e) with Holm-Sidak's multiple comparisons test.

FIGS. 9a-g. Molecular and functional characterization of GINS organoids derived from multiple donors. a, Schematic diagram and representative images of cells at key stages in GINS organoid formation. Ngn3ER-hGSCs: human gastric stem cells that incorporated a Ngn3 and estrogen receptor (ER) fusion gene (Ngn3ER); 4-OH-TAM: 4-OH Tamoxifen; Lenti-CMVPM, lentiviral integration of a polycistronic Pdx1-Mafa co-expression cassette. b, Representative immunofluorescent staining of corpus GINS organoids derived from three different donors, and co-localization of INS and CPPT in GINS cells. a, b, Repeated independently 3 times with similar results. c, To assess CPPT+ mono-hormonal cells, a cocktail of GCG, SST and GHRL antibodies were stained together with CPPT in day-21 GINS organoids. Right panel shows immunofluorescent staining of CPPT (red) and a combination of GCG, GHRL and SST (green). left panel shows quantification of mono-hormonal CPPT+ cells (n=10 organoids from donor #6). d, Relative expression of endocrine hormone genes including INS, GCG, SST, and GHRL in day-18 GINS organoids derived from four different donors in comparison with human islets. n=4 (donor #6) or 3 (donor #7, #9, #10, or islet donors) separate batches of samples for each donor. e, Relative expression of key β-cell markers in GINS organoids derived from different donors in comparison with human islets. n=4 (donor #6) or 3 (donor #7, #9, #10, or islet donors) separate batches of samples for each donor. f, Glucose-stimulated insulin secretion of GINS organoids at different time points (n=3 independent samples from donor #6 for each batch of differentiation) or donor #9 (n=3 independent samples, day-18). g, Insulin secretion of day-18 GINS organoids from donor #6 incubated with the indicated concentrations of glucose with or without 10 nM glibenclamide (Glib) or 0.5 mM diazoxide (Dzx) as indicated (n=4 independent samples). c-g, Data presented as mean±s.d.; one-way ANOVA (d, e, g) or repeated-measures two-way ANOVA with Holm-Sidak's multiple comparisons test for different time points (f) or one-tailed paired t-test for donor #9 (f).

FIGS. 10a-e. Characterizing GINS organoid cells with scRNA-seq. a, Top UMAP, cells sampled from hGSC cultures (blue, n=1, donor #6) or GIN organoids (red, n=1, donor #6); middle UMAP, hGSC cultures included both stem cells (stem) and mucus-secreting cells (mucus) spontaneously differentiated from hGSCs. GINS organoids contained four endocrine cell types. Cells are colored according to cell types; bottom UMAP, relative expression of cell type specific markers. b, Relative expression of endocrine cell type-specific markers. The shading displays scaled average gene expression, and diameter denotes fractional expression. c, Comparison of GINS β-like cells (n=2 independent batch of organoids, one representative batch shown, donor #6) and islet β-cells (n=4 independent donors, an integration of all samples) in expression profiles of key genes for β-cell function, identity, metabolism, and exocytosis. MODY: Maturity Onset Diabetes of the Young. d, Relative expression of disallowed genes in the indicated cell types (n=1, donor #6). e, Violin plots showing the expression levels of proliferative markers in the indicated cell types (n=1, donor #6).

FIGS. 11a-f. scRNA-seq comparison of GINS organoids derived from human antrum vs corpus stomach. a, Diagram of human stomach. b, Immunofluorescence of antral GINS organoid (donor #6) stained for CPPT and MAFA. Repeated independently 5 times with similar results. c, Comparison of corpus and antral GINS organoids (both from donor #6) in the expression of β-cell marker genes. n=3 independent experiments. Data presented as mean±s.d.; one-way ANOVA with Dunnett multiple comparisons test comparing GINS with islets. d, t-distributed stochastic neighbor embedding (t-SNE) plots of integrated corpus and antral GINS organoids. Cells are colored according to cell types. G-like: G-like cells that expressed gastrin (GAST). Pie charts indicate cell-type proportions. e, Comparison of antral and corpus GINS β-like cells expression profiles of key genes for β-cell function and identity. Red, antral GINS β-like cells; Blue, corpus GINS β-like cells. f, Glucose-stimulated insulin secretion of antral GINS organoids at different time points (days post differentiation) or from different batches. n=4 independent groups of GINS organoids for the time course GSIS. n=5 independent groups of GINS organoids for batch 1, n=4 independent groups of GINS organoids for batch 2 and 3. Two-way repeated-measures ANOVA with Holm-Sidak's multiple comparisons test.

FIGS. 12a-b. GINS organoids are not fully mature. a, Volcano plot comparing gene expression of GINS β-like cells (n=2 independent batch of organoids, one representative batch is shown, donor #6) versus islet β cells (n=4 independent donors, an integration of all samples) identified in FIG. 3a. The number of differentially expressed genes (DEGs) enriched in either cell group is shown in the plot. Threshold of DEGs: adjusted-P<0.01 and log 2 fold-change>1. P-value calculated by Wilcoxon Rank Sum test and adjusted based on Bonferroni correction. b, Gene Ontology (GO) analysis of DEGs enriched in GINS β-like cells (blue) or islet β-cells (red). P-value calculated by hypergeometric distribution followed by Benjamini-Hochberg adjustment.

FIGS. 13a-h. Phenotypic characterization of GINS grafts. a, Quantification from immunofluorescent staining of marker proteins, n=3 independent experiments. Representative image showing co-expression of INS and CPPT in the GINS graft. b, Electron microscopy imaging of GINS graft. The electron-dense core granules were partially condensed. Repeated independently 3 times with similar results. c, SLC30A8 relative expression levels in GINS organoids, GINS grafts, and human islets. n=3 independent groups of GINS organoids, independent GINS grafts from different mice, or independent human islets from different donors. Data presented as mean±s.d.; one-way ANOVA with Dunnett multiple comparisons test comparing GINS with islets. d, Images of the kidney from mice transplanted with GINS cells on day 0 and day 110 post transplantation. e, mCherry-labeled hGSCs (0.5×106) transplanted under the renal capsule and visualized under fluorescent microscope on day 0 and day 80 post transplantation. No Cherry+ cells were found at day 80. Repeated independently 5 times with similar results. f, tSNE projection of integrated GINS organoids and grafts. Cells are colored according to cell types. Horizontal bars indicate cell type ratios. g, Violin plots showing the expression levels of select ribonucleoproteins. h, Relative expression of select genes in the pathways elevated in cultured GINS β-like cells compared with human islet-β cells.

FIGS. 14a-c. Dynamic gene and signaling pathway activations in hGSC differentiation to GINS organoid. a, Relative expression levels of cell type-specific markers in UMAP. b, Expression of select genes shown along pseudotime in GINS organoid formation. Each dot represents a cell. a, b, n=1, donor #6. c, Heat map showing transcription factor expression clusters along the pseudotime trajectory from hGSCs to GINS organoid cells (n=1, donor #6) and islet β cells (n=1, donor #1). Density plot on the top showing cell populations along pseudotime. Stem: hGSCs; Endo 1: endocrine progenitors 1; Endo 2: endocrine progenitors 2; GINS pre: GINS precursors.

FIGS. 15a-c. Characterization of the developmental path of GINS organoids. a, Heatmap showing waves of transcription factor regulon activations. Key regulons are labeled on the right with the number of their predicted target genes. Stem: hGSCs; Endo 1: endocrine progenitors 1; Endo 2: endocrine progenitors 2; GINS pre: GINS precursors. b, Select regulon activity overlaid on UMAP. c, RNA velocity and pseudotime trajectory analysis in UMAP showing the developmental path from GINS precursors to endocrine cells in GINS organoids. a, b, n=1, donor #6.

### DETAILED DESCRIPTION

This disclosure describes a robust protocol to induce cultured human gastric stem and progenitor cells (hGSCs) to differentiate into islet-like organoids at high efficiency, containing approximately 70% β-like cells and other islet-like endocrine populations. Human gastric insulin secreting (GINS) organoids developed herein have been shown to exhibit glucose responsiveness, secret human insulin and reverse diabetes in mice, and are stable upon transplantation for 6 months or longer. No proliferative cells are detected in transplanted GINS organoids whereas hGSCs perish upon engraftment. Human GINS cells and GINS organoids prepared herein thus possess favorable attributes as a potential transplantable therapeutic. Accordingly, provided herein after methods for producing human gastric insulin-secreting (GINS) cells, methods of producing human gastric insulin-secreting (GINS) organoids, human gastric insulin-secreting (GINS) cells prepared by the present methods, human gastric insulin-secreting (GINS) organoids obtained herein, and therapeutic methods by using the human GINS cells or organoids prepared herein.

### Gastric Stem and Progenitor Cells

“Gastric stem and progenitor cells”, as used herein, refer to cells typically known as gastric stem cells, and cells that have the same structural and functional characteristics as gastric stem cells but may be referred to by others under different names (e.g., gastric progenitor cells). Gastric stem cells represent an adult stem cell population residing in and/or obtainable from the stomach tissues with the ability of self-renewal and multi-potency, which enables efficient stomach epithelium regeneration and repair. Under physiological conditions, gastric epithelial cells undergo continuous dynamic renewal. Consequently, gastric stem cells are essential for the regeneration of lost or damaged cells in stomach mucosa. Gastric stem cells may include: (i) stem cells in the antrum characterized by Lgr5⁺, CCKR2⁺, Axin2⁺ and AQP5⁺, (ii) Mist1⁺ cells and Troy⁺ mature chief cells in the corpus, and (iii) Sox2, eR1, Lrig1, Bmi1-marked cells in both the antrum and the corpus section of the stomach (Xiao et. al, 2020, Frontiers in Cell and Developmental Biology, 8). In some embodiments, gastric stem and progenitor cells used herein express SOX9 and KI67 markers. In some embodiments, gastric stem and progenitor cells used herein express SOX9, Lgr5 and KI67 markers, but negative for Cdx2 (an intestine marker). Gastric stem and progenitor cells can be prepared from human gastric tissues using methodologies established in the art, e.g., Wang, et.al., 2015, Nature 522, 173-178; Sato, et.al., 2009, Nature 459, 262-265; Sato, et.al., 2011, Gastroenterology 141, 1762-1772. Preparation of human gastric stem and progenitor cells is also described hereinbelow and illustrated in the Examples section herein. Briefly, human gastric tissues can be cut into small pieces and incubated with medium containing collagenase type IV until most of the glandular cells are released and appear in solution as clusters. The cells can then be collected and resuspended in a human gastric stem cell culture medium (hGSC medium), then seeded and cultured on mitomycin-C-inactivated mouse embryonic fibroblasts. In some embodiments, a hGSC medium comprises R-spondin (e.g., R-spondin-2, or alternatively R-spondin-1 and R-spondin-3), EGF, and DMH1 (or any other inhibitors of BMP signaling, for instance, Noggin). In some embodiments, the EGF concentration is 10-100 ng/ml, the DMH1 concentration is 0.5-2 μM. R-spondin-2 can be provided via a conditioned medium. In specific embodiments, a hSGC medium is described as basal medium composed of 66.7% DMEM, 33.3% F12K supplemented with 18% FBS, 10% R-Spondin-2 conditioned medium, 10 mM nicotinamide, 25 μM primocin, 1 μM A8301, 5 μg/mL insulin, 10 μM Y-27632, 1 μM DMH1, 50 ng/mL EGF and 2 μM T3. It typically takes 5-10 days for gastric stem cell colonies to emerge, visible under a microscope). hGSC colonies, in an undifferentiated state, generally appear as round colonies. The cells are compact with high nucleus to cytoplasmic ratio. When the colonies get larger, they become more irregular in shape and spontaneous differentiation will occur in the center of the colonies where the cells will become larger and show lower nucleus to cytoplasmic ratio. Higher-lower nucleus/cytoplastic ratio is based on comparing stem cells and differentiated gastric cells. Cultured Antrum and corpus GSCs express common markers including Sox9, Lgr5 and Ki67, and no Cdx2 (an intestine marker). The assessment can be made by one or a combination of methods including qPCR, scRNA-seq and immunohistochemistry.

### Insulin Secreting Pancreatic β-Cells

The pancreatic β-cell plays a key role in glucose homeostasis by secreting insulin, the only hormone capable of lowering the blood glucose concentration. The pancreatic β-cells are endocrine cells that synthetize, store, and release insulin, the anti-hyperglycemic hormone that antagonizes glucagon, growth hormone, glucocorticosteroids, epinephrine, and other hyperglycemic hormones, to maintain circulating glucose concentrations within a narrow physiologic range. The pancreatic islets are endocrine micro-organs that are embedded in the exocrine parenchyma of the pancreas. The mature pancreatic islet consists of several types of endocrine cells. The most important are the insulin-secreting β-cells (which make up 50% of cells in human islets and 75% in the mouse), the glucagon-releasing α-cells (35-40% in human and 15-20% in mice), and the somatostatin-releasing δ-cells (10-15% in human and ˜5% in the mouse). The β-cells are the principal component of the pancreatic islets in all species. The β-cells markers include G6PC2, GCK, ABCC8, NKX2-2, PCSK1, PAX6, PDX1, NKX6-1, and NEUROD1. The β-cells are polygonal cells, with an average diameter of 13-18 m that possess ˜10,000 secretory granules, each containing up to 8-9 fg insulin (1.6-1.8 amol insulin). This corresponds to an intragranular insulin concentration of ˜100 mM. Insulin is stored in crystalline form in the secretory vesicles as a Zn2-insulin complex and accounts for 5-10% of the total protein content of the β-cell, more than any other protein. It is released by regulated exocytosis. Only a small fraction of the secretory granules (<1%/h) undergo exocytosis even at high glucose concentrations (Rorsman et.al., 2018, Physiol Rev., 98(1), 117-214).

### Gastric Insulin-Secreting (GINS) Cells

Human GINS cells have been prepared herein and shown herein to express insulin and other β-cell genes at similar levels as pancreatic β-cells, have comparable insulin content, and exhibit static and dynamic glucose-stimulated insulin secretion (GSIS). Cultured GINS cells have also been shown herein to respond to stimulation with the clinical anti-diabetic drugs liraglutide and Glibenclamide and the anti-hypoglycemia drug Diazoxide.

Upon transplantation into mouse models of diabetes (induced by chemical ablation of endogenous pancreatic β-cells), human GINS cells prepared herein have also been shown to secret human insulin and c-peptide into circulation, respond to high glucose challenge, rapidly suppress hyperglycemia (within 2 days of grafting). and maintain normoglycemia for over 100 days until graft removal, upon which hyperglycemia returned. Thus, human GINS cells have demonstrable therapeutic properties in a diabetes setting.

The present disclosure is the first to show that insulin-secreting and glucose-responsive cells can be made from human stomach tissues.

Human GINS cells are unique therapeutic entities, akin to novel small molecule compounds or antibodies. The molecular, physiological and transplantation data demonstrate that human GINS cells are glucose-responsive and insulin-secreting, able to reverse diabetes and maintain normoglycemia for extended period. Grafted human GINS cells do not proliferate and show no signs of tumor formation.

The human GINS cells described herein produce high level insulin, e.g., levels comparable to primary human islets. Although human GINS cells resemble pancreatic 3-cells in molecular and functional properties, they are not identical. There are notable differences between them:
(a) human GINS cells do not express certain key 3-cell markers, including NKX6-1 and GAD65 (GAD65 is a major autoantigen targeted by autoantibodies in T1D patients);
(b) electron microscopy studies showed that the insulin granule morphology of human GINS cells differs from that of islet β-cells (for example, electron microscopy study has shown that the electron-dense insulin granules of the GINS cells are not fully condensed); and/or
(c) human GINS cells retain residual gastric gene expression, as measured, e.g., by scRNA-seq. Table 1 shows the list of β-cell-specific genes and gastric-specific genes. A gastric score can be calculated based on the expression levels of gastric-specific genes, e.g., using a published statistical method (Tirosh et al, 2016, Science). In some embodiments, the gastric score is calculated based on expression levels of some of the 868 stomach-specific genes listed in Table 1, e.g., 50, 100, 200, 300, 400, 500, 600, 700, or 800 genes. In some embodiments, the gastric score is based on expression levels of all of the 868 stomach-specific genes listed in Table 1. The gastric score in GINS cells is statistically higher than pancreatic (3-cells, but much lower than that of bona fide gastric cells. The human GINS cells thus are considered to retain residual gastric gene expression.

GINS cells do not exist in nature as stomach tissues never make β-cells or insulin-secreting cells.

“A population of GINS cells” described herein refers to a substantially purified population of GINS cells, i.e., a cell population enriched in GINS cells, e.g., at least 50%-60% of the cell population are GINS cells, at least 70%, 80%, 90% of the cell population are GINS cells. GINS cells can be made by the methods described herein.

In one aspect, disclosed herein is a population of human gastric insulin-secreting (GINS) cells, where the GINS cells are glucose-responsive and insulin-secreting; do not express certain 3-cell markers such as NKX6-1 and GAD65; secrete insulin but having a granule morphology different from that of islet β-cells and retain residual gastric gene expression.

In some embodiments, the residual gastric gene expression is determined by single cell RNA sequencing. In some embodiments, the granule morphology of the secreted insulin is determined by electron microscopy.

In some embodiments, the residual gastric gene expression is determined by single cell RNA sequencing. In some embodiments, the granule morphology of the secreted insulin is determined by electron microscopy.

### Gastric Insulin-Secreting (GINS) Organoids

Organoids are tiny, self-organized three-dimensional tissue cultures that are derived from stem and progenitor cells. Such cultures can be crafted to replicate much of the complexity of an organ, or to express selected aspects of it like producing only certain types of cells. An organoid mimics its corresponding in vivo organ, such that it can be used to study aspects of that organ in the tissue culture dish.

This disclosure provides a method to direct cultured human gastric stem cells (hGSCs) to generate pancreatic islet-like organoids containing long-lived gastric insulin-secreting (GINS) cells that resemble pancreatic β-cells and able to reverse diabetes after transplantation. Cultured GINS cells spontaneously aggregate into islet-like organoids and acquire glucose-stimulated insulin secretion (GSIS).

GINS organoids like GINS cells are unique therapeutic entities, akin to novel small molecule compounds or antibodies. The molecular, physiological and transplantation data demonstrate that GINS organoids are glucose-responsive and insulin-secreting, able to reverse diabetes and maintain normoglycemia for extended period. Grafted GINS organoids do not proliferate and show no signs of tumor formation. GINS organoids can be made by the methods described herein.

It has been demonstrated herein through single cell RNA sequencing (scRNA-seq) that GINS organoids prepared herein contain four endocrine cell types that closely resembled the four major human islet cells, namely, β, α, δ, and ε cells. Consistent with their functional competence, GINS cells are shown to express key genes involved in β-cell identity, metabolism, insulin synthesis and secretion, and ion channel activities. Molecular scorecards of s-cells (1,034 β-cell-specific genes) and gastric cells (868 stomach-specific genes) benchmarked from published human scRNA-seq data have been applied to further assess the identity of GINS cells. GINS cells have been shown to score high in β-cells and low in gastric signature, similar to islet β-cells, although GINS cells possess residual gastric signature. The gastric score, calculated based on the expression levels of gastric specific genes (e.g., the 868 genes in Table 1), is statistically higher in GINS cells than pancreatic β-cells, but much lower than that of bona fide gastric cells. The GINS cells thus are considered to retain residual gastric gene expression. Table 1 shows the list of β-cell-specific genes and gastric-specific genes.

In one aspect, disclosed herein is a preparation of human gastric insulin-secreting (GINS) organoids, wherein the GINS organoids comprise GINS cells that are glucose-responsive and insulin-secreting; do not express R-cell markers such as NKX6-1 and GAD65; secrete insulin but having a granule morphology different from that of islet β-cells and retain residual gastric gene expression.

### Methods of Generation of Human Gastric Insulin-Secreting (GINS) Cells

Human GINS cells can be generated by inducing expression of genetic factors NGN3, PDX1 and MAFA in cultured human gastric stem and progenitor cells.

In some embodiments, a method of producing human gastric insulin-secreting (GINS) cells comprises: obtaining and culturing gastric stem and progenitor cells from a gastric tissue sample of a human subject; manipulating the gastric stem and progenitor cells to cause the gastric stem and progenitor cells to express a NGN3 factor, a PDX1 factor, and a MAFA factor; and culturing the manipulated cells in a serum free medium to obtain the GTNS cells, wherein the GINS cells are insulin-secreting and glucose-responsive.

Human gastric stem and progenitor cells can be prepared from a gastric tissue sample obtained from a human subject, e.g., a biopsy sample from a human gastric tissue. Gastric stem and progenitor cells can be prepared from human gastric tissues using methodologies established in the art, e.g., Wang, et.al., 2015, Nature 522, 173-178; Sato, et.al., 2009, Nature 459, 262-265; Sato, et.al., 2011, Gastroenterology 141, 1762-1772. Preparation of human gastric stem and progenitor cells is also described hereinbelow and illustrated in the Examples section herein. In exemplary embodiments, human gastric tissues can be cut into small pieces and incubated with medium containing collagenase type IV until most of the glandular cells are released and appear in solution as clusters. The cells can then be collected by centrifugation and resuspended in human gastric stem cell culture medium (hGSC medium) and seeded on mitomycin-C-inactivated mouse embryonic fibroblasts. In some embodiments, a hGSC medium comprises R-spondin (e.g., R-spondin-2, or alternatively R-spondin-1 and R-spondin-3), EGF, and DMH1 (or any other inhibitors of BMP signaling, for instance, Noggin). In some embodiments, the EGF concentration is 10-100 ng/ml, the DMH1 concentration is 0.5-2 μM. R-spondin-2 can be provided via a conditioned medium. In specific embodiments, a hSGC medium is described as basal medium composed of 66.7% DMEM, 33.3% F12K supplemented with 18% FBS, 10% R-Spondin-2 conditioned medium, 10 mM nicotinamide, 25 μM primocin, 1 μM A8301, 5 μg/mL insulin, 10 μM Y-27632, 1 μM DMH1, 50 ng/mL EGF and 2 μM T3. It typically takes 5-10 days for gastric stem cell colonies to emerge, visible under a microscope). hGSC colonies, in an undifferentiated state, generally appear as round colonies. The cells are compact with high nucleus to cytoplasmic ratio. When the colonies get larger, they become more irregular in shape and spontaneous differentiation will occur in the center of the colonies where the cells will become larger and show lower nucleus to cytoplasmic ratio. Higher-lower nucleus/cytoplastic ratio is based on comparing stem cells and differentiated gastric cells. Cultured Antrum and corpus GSCs express common markers including Sox9, Lgr5 and Ki67, and no Cdx2 (an intestine marker). The assessment can be made by one or a combination of methods including qPCR, scRNA-seq and immunohistochemistry. Each biopsy-sized gastric sample typically yields 30-40 primary colonies, which can be amplified to >10⁹ gastric stem and progenitor cells (GSCs) within 2 months. Cultured hGSCs continue to express the stomach stem/progenitor marker SOX9 and the proliferative marker K167 after many passages. hGSCs are typically maintained at 37° C. in a 7.5% CO₂ incubator. Culture medium is changed every 2-3 days and hGSC colonies are split every 4-6 days at a ratio between 1:3 and 1:5.

To derive GINS, the gastric stem and progenitor cells are manipulated to express a NGN3 factor, a PDX1 factor, and a MAFA factor (or collectively “NPM factors”).

The term “express” or “expression”, when used herein in connection with a transcription factor, means manifestation of the function of the transcription factor. Thus, in this disclosure, expression of a transcription factor can, in some instances, may be aligned and consistent with expression of a gene encoding the transcription factor; while in other instances, expression of a transcription factor does not necessarily align with gene expression. For example, an exogenous nucleic acid encoding a transcription factor can be introduced into a desired cell, transcribed to make an mRNA which is then translated to make the transcription factor; yet if the activity of the transcription factor is inhibited (e.g., as a result of a design of the transcription factor being fused to an estrogen receptor), it is understood herein that the transcription factor is not “expressed” for purpose of this disclosure (i.e., its function is not manifested) until the inhibition is removed.

In some embodiments, expression of the factors can be induced by exogenously introducing one or more viral vectors, or mRNA molecules encoding the factors, or genetic engineering (e.g., using CRISPR or TALEN). In some embodiments, CRISPR or TALEN can be used to integrate one or more expression cassettes into the genome of the gastric cells at a specific locus, for instance, the AAVS1 safe harbor locus. In some embodiments, one or more lentiviral vectors are used. In some embodiments, one or more AAV vectors are used. In some embodiments, expression of the endogenous factors can be induced by using small molecules. Regardless of the induction method, the resulting GINS cells and GINS organoids will have similar molecular and functional properties.

In some embodiments, the NGN3 factor is expressed for at least 1 day, e.g., for 2 days. In some embodiments, the PDX1 factor and the MAFA factor are stably expressed. In some embodiments, the expression of the NGN3 factor is transient, followed by stable expression of the PDX1 factor and the MAFA factor. In some embodiments, the expression of the NGN3 factor lasts for 1-3 days (e.g., 2 days), followed by stable expression of the PDX1 factor and the MAFA factor (e.g., for at least 2 to 6 days, i.e., 2 days, 3 days, 4 days, 5 days, 6 days, or longer).

“Transient expression” of a transcription factor refers to manifestation of the function of the transcription factor for a short time. Transient expression can be achieved by temporary modulation of the function of a protein. In some embodiments, for transient expression of NGN3, a Ngn3 and estrogen receptor (ER) fusion gene (Ngn3ER) is incorporated into the hGSCs by lentivirus. While the gene expression of the fusion construct is constitutive, expression of the NGN3 function (ability to bind genomic DNA and activate endocrine gene expression) is suppressed by the ER protein. Addition of 40H-TAM relieves this protein-protein inhibition and allows the “expression” of Ngn3 function (to bind DNA and activate gene expression). Thus, the temporary/transient expression of Ngn3 can be achieved by 4OH-Tamoxifen treatment of cultured Ngn3ER-hGSCs for 1-3 days (e.g., 2 days), which initiate hGSC differentiation. Detailed methodology is illustrated in the Examples section herein.

Stable expression of a transcription factor refers to the constitutive or persistent expression of the function of the transcription factor. In some embodiments, stable expression of a transcription factor in a desired cell is achieved by transducing the cell with a gene of interest encoding the transcription factor. In some embodiments, stable PDX1 and MAFA expression is accomplished by lentiviral integration of expression cassette (e.g., a Pdx1-Mafa co-expression cassette) into the hGSCs. In some embodiments stable PDX1 and MAFA expression can be achieved using vectors that do not integrate into the host genome, for instance, AAV vectors. Detailed methodology is illustrated in the Examples section herein. In some embodiments, stable expression is to use CRISPR-activators, delivered by a transient vector system (such as mRNA) to directly activate endogenous Pdx1 and Mafa for constitutive expression.

In some embodiments, cultured hGSCs are transduced with viral vectors (e.g., lentiviral) encoding the NPM factors. The transduced cells are cultured in hGSC medium and are induced (e.g., by treatment with 4-OA TAM) to transiently express NGN3 for 1-3 days (e.g., 2 days). The culture medium is then changed to serum-free medium and the cells are induced (e.g., through doxycycline added to the medium) to stably express PDX1 and MAFA for at least 2 to 6 days, i.e., 2 days, 3 days, 4 days, 5 days, 6 days, or longer. In some exemplary embodiments, the serum-free medium comprises 75% GINS medium and 25% hGSC medium. In some exemplary embodiments, GINS medium can be formulated as the follows: advanced DMEM/F12 supplemented with 10 mM HEPES, 1× GlutaMAX, 25 μM Primocin, 500 μM NAC, 1× B-27, 1× N-2, 10 mM Nicotinamide, 1 μM A8301, and 10 μM Y-27632; and the hGSC medium can be formulated as: basal medium composed of 66.7% DMEM, 33.3% F12K supplemented with 18% FBS, 10% R-Spondin-2 conditioned medium, 10 mM nicotinamide, 25 μM primocin, 1 μM A8301, 5 μg/mL insulin, 10 μM Y-27632, 1 μM DMH1, 50 ng/mL EGF and 2 μM T3.

### Exogenous Nucleic Acids

Exogenous nucleic acids are nucleic acids originating outside an organism that has been introduced into the organism. Exogenous nucleic acids may enter the nucleus, where some are absorbed and/or blocked by heterochromatin and others integrate into chromosomes. Examples of exogenous nucleic acids suitable for use to manipulate the gastric stem and progenitor cells to express transcription factors include nucleic acid vectors such as plasmids or viral vectors, or mRNAs including modified mRNAs.

Modified RNA (modRNA)

A nucleoside is a molecule including a nitrogenous base (i.e., a nucleobase) linked to a pentose (e.g., deoxyribose or ribose) sugar. Nitrogenous bases which form nucleosides include adenine, guanine, cytosine, 5-methyl cytosine, uracil, and thymine. Suitable ribonucleosides (which comprise ribose as the pentose sugar) include, e.g., adenosine (A), guanosine (G), 5-methyluridine (m5U), uridine (U), and cytidine (C). Nucleotides are molecules including a nucleoside (e.g., a ribonucleoside) and a phosphate group. Ribonucleotides include, e.g., adenosine monophosphate, adenosine diphosphate, adenosine triphosphate, guanosine monophosphate, guanosine diphosphate, guanosine triphosphate, cytidine monophosphate, cytidine diphosphate, cytidine triphosphate, uridine monophosphate, uridine diphosphate, uridine triphosphate, and derivatives thereof.

Modified RNA, or modRNA, is a synthetic modified RNA that can be used for expression of a gene of interest. Chemical modifications to a ribonucleotide included in modRNA may stabilize an RNA molecule, blunt an immune response, or enhance transcription. Additionally, unlike delivery of protein agents directly to a cell, which can activate the immune system, the delivery of modRNA can be achieved without immune impact. For example, substitution of uridine and cytidine with pseudouridine or N1-methylpseudouridine and 5-methylcytidine, respectively, drastically reduces the immune response elicited from exogenous RNA without such substitutions. Stability and translational efficiency from an RNA molecule may also be increased by including a 3′-O-Me-m7G(5′)ppp(5′)G Anti Reverse Cap Analog (ARCA) at the 5′ end of the RNA molecule.

modRNA may encompass an RNA molecule with at least uridine substituted with pseudouridine. modRNA may encompass an RNA molecule with at least cytidine substituted with 5-methylcytidine. modRNA may encompass an RNA molecule including the modified nucleoside 5-methylcytidine (5mC). modRNA may encompass an RNA molecule including the modified nucleoside 2-Thiouridine-5′-Triphosphate (2-thio ψU). modRNA may encompass an RNA molecule with at least the modified nucleoside 1-Methylpseudouridine-5′-Triphosphate (1-mψU). modRNA may encompass an RNA molecule with at least the modified nucleoside N1-methyl-pseudouridine (N1mΨ) substituted for uridine. modRNA may encompass an RNA molecule wherein at least 5′ triphosphates are removed. modRNA may encompass an RNA molecule wherein at least a 3′-O-Me-m7G(5′)ppp(5′)G Anti Reverse Cap Analog (ARCA) cap or C₃₂H₄₃N₁₅O₂₄P₄ CleanCap Reagent AG is included in a 5′ untranslated regions of the RNA molecule.

modRNAs may be prepared by in vitro transcription. modRNA may be in vitro transcribed, e.g., from a linear DNA template using one or more reagents selected from a cap analog, guanosine triphosphate, adenosine triphosphate, cytidine triphosphate, uridine triphosphate, and derivatives thereof. A cap analog may be selected from Anti-Reverse Cap Analog (ARCA) 3′-O-Me-m7G(5′)ppp(5′)G, standard cap analog m7G(5′)ppp(5′)G, unmethylated cap analog G(5′)ppp(5′)G, methylated cap analog for A+1 sites m7G(5′)ppp(5′)A, and unmethylated cap analog for A+1 sites G(5′)ppp(5′)A. In certain examples, a cap analog is Anti-Reverse Cap Analog (ARCA) 3′-O-Me-m7G(5′)ppp(5′)G. According to some examples, modRNA may be in vitro transcribed from a plasmid template using one or more reagents selected from 3′-O-Me-m7G(5′)ppp(5′)G, guanosine triphosphate, adenosine triphosphate, cytidine triphosphate, N1-methylpseudouridine-5-triphosphate, and any one or more of the aforementioned examples of modRNA, or others, without limitation and in any combination. Additional suitable modifications to a modRNA or mRNA molecule are well known in the art (e.g., U.S. Pat. No. 8,278,036 to Kariko et al.; U.S. Pat. No. 10,086,043 to Chien et al.; U.S. Patent Application Publication No. 2019/0203226 to Zangi et al.; and U.S. Patent Application Publication No. 2018/0353618 to Burkhardt et al.; all of which are hereby incorporated by reference in their entirety). In some embodiments, the nucleoside that is modified in the modRNA is a uridine (U), a cytidine (C), an adenine (A), or guanine (G). The modified nucleoside can be, for example, m⁵C (5-methylcytidine), m⁶A (N⁶-methyladenosine), s²U (2-thiouridien), ψ (pseudouridine), or Um (2-O-methyluridine). Some exemplary chemical modifications of nucleosides in the modRNA molecule may further include, for example and without limitation, pyridine-4-one ribonucleoside, 5-aza-uridine, 2-thio-5-aza uridine, 2-thiouridine, 4-thio pseudouridine, 2-thio pseudouridine, 5-hydroxyuridine, 3-methyluridine, 5-carboxymethyl uridine, 1-carboxymethyl pseudouridine, 5-propynyl uridine, 1-propynyl pseudouridine, 5-taurinomethyluridine, 1-taurinomethyl pseudouridine, 5-taurinomethyl-2-thio uridine, 1-taurinomethyl-4-thio uridine, 5-methyl uridine, 1-methyl pseudouridine, 4-thio-1-methyl pseudouridine, 2-thio-1-methyl pseudouridine, 1-methyl-1-deaza pseudouridine, 2-thio-1-methyl-1-deaza pseudouridine, dihydrouridine, dihydropseudouridine, 2-thio dihydrouridine, 2-thio dihydropseudouridine, 2-methoxyuridine, 2-methoxy-4-thio uridine, 4-methoxy pseudouridine, 4-methoxy-2-thio pseudouridine, 5-aza cytidine, pseudoisocytidine, 3-methyl cytidine, N4-acetylcytidine, 5-formylcytidine, N4-methylcytidine, 5-hydroxymethylcytidine, 1-methyl pseudoisocytidine, pyrrolo-cytidine, pyrrolo-pseudoisocytidine, 2-thio cytidine, 2-thio-5-methyl cytidine, 4-thio pseudoisocytidine, 4-thio-1-methyl pseudoisocytidine, 4-thio-1-methyl-1-deaza pseudoisocytidine, 1-methyl-1-deaza pseudoisocytidine, zebularine, 5-aza zebularine, 5-methyl zebularine, 5-aza-2-thio zebularine, 2-thio zebularine, 2-methoxy cytidine, 2-methoxy-5-methyl cytidine, 4-methoxy pseudoisocytidine, 4-methoxy-1-methyl pseudoisocytidine, 2-aminopurine, 2,6-diaminopurine, 7-deaza adenine, 7-deaza-8-aza adenine, 7-deaza-2-aminopurine, 7-deaza-8-aza-2-aminopurine, 7-deaza-2,6-diaminopurine, 7-deaza-8-aza-2,6-diaminopurine, 1-methyladenosine, N⁶-methyladenosine, N⁶-isopentenyladenosine, N⁶-(cis-hydroxyisopentenyl) adenosine, 2-methylthio-N⁶-(cis-hydroxyisopentenyl) adenosine, N⁶-glycinylcarbamoyladenosine, N⁶-threonylcarbamoyladenosine, 2-methylthio-N⁶-threonyl carbamoyladenosine, N⁶,N⁶-dimethyladenosine, 7-methyladenine, 2-methylthio adenine, 2-methoxy adenine, inosine, 1-methyl inosine, wyosine, wybutosine, 7-deaza guanosine, 7-deaza-8-aza guanosine, 6-thio guanosine, 6-thio-7-deaza guanosine, 6-thio-7-deaza-8-aza guanosine, 7-methyl guanosine, 6-thio-7-methyl guanosine, 7-methylinosine, 6-methoxy guanosine, 1-methylguanosine, N²-methylguanosine, N²,N²-dimethylguanosine, 8-oxo guanosine, 7-methyl-8-oxo guanosine, 1-methyl-6-thio guanosine, N²-methyl-6-thio guanosine, or N²,N²-dimethyl-6-thio guanosine.

In some embodiments, modifications made to the modRNA are independently selected from 5-methylcytosine, pseudouridine, and 1-methylpseudouridine.

In some embodiments, the modRNA comprises a modified uracil selected from the group consisting of pseudouridine (ψ), pyridine-4-one ribonucleoside, 5-aza uridine, 6-aza uridine, 2-thio-5-aza uridine, 2-thio uridine (s2U), 4-thio uridine (s4U), 4-thio pseudouridine, 2-thio pseudouridine, 5-hydroxy uridine (ho⁵U), 5-aminoallyl uridine, 5-halo uridine (e.g., 5-iodom uridine or 5-bromo uridine), 3-methyl uridine (m³U), 5-methoxy uridine (mo⁵U), uridine 5-oxyacetic acid (cmo⁵U), uridine 5-oxyacetic acid methyl ester (mcmo⁵U), 5-carboxymethyl uridine (cm⁵U), 1-carboxymethyl pseudouridine, 5-carboxyhydroxymethyl uridine (chm⁵U), 5-carboxyhydroxymethyl uridine methyl ester (mchm⁵U), 5-methoxycarbonylmethyl uridine (mcm⁵U), 5-methoxycarbonylmethyl-2-thio uridine (mcm⁵s2U), 5-aminomethyl-2-thio uridine (nm⁵s2U), 5-methylaminomethyl uridine (mnm⁵U), 5-methylaminomethyl-2-thio uridine (mnm⁵s2U), 5-methylaminomethyl-2-seleno uridine (mnm⁵se²U), 5-carbamoylmethyl uridine (ncm⁵U), 5-carboxymethylaminomethyl uridine (cmnm⁵U), 5-carboxymethylaminomethyl-2-thio uridine (cmnm⁵s2U), 5-propynyl uridine, 1-propynyl pseudouridine, 5-taurinomethyl uridine (τcm⁵U), 1-taurinomethyl pseudouridine, 5-taurinomethyl-2-thio uridine (^{TM5}s2U), 1-taurinomethyl-4-thio pseudouridine, 5-methyl uridine (m⁵U, e.g., having the nucleobase deoxythymine), 1-methyl pseudouridine (m¹ψ), 5-methyl-2-thio uridine (m⁵s2U), 1-methyl-4-thio pseudouridine (m₁s⁴ψ), 4-thio-1-methyl pseudouridine, 3-methyl pseudouridine (m³ψ), 2-thio-1-methyl pseudouridine, 1-methyl-1-deaza pseudouridine, 2-thio-1-methyl-1-deaza pseudouridine, dihydrouridine (D), dihydropseudouridine, 5,6-dihydrouridine, 5-methyl dihydrouridine (m⁵D), 2-thio dihydrouridine, 2-thio dihydropseudouridine, 2-methoxy uridine, 2-methoxy-4-thio uridine, 4-methoxy pseudouridine, 4-methoxy-2-thio pseudouridine, N¹-methyl pseudouridine, 3-(3-amino-3-carboxypropyl) uridine (acp³U), 1-methyl-3-(3-amino-3-carboxypropyl) pseudouridine (acp³ψ), 5-(isopentenylaminomethyl) uridine (inm⁵U), 5-(isopentenylaminomethyl)-2-thio uridine (inm⁵s2U), α-thio uridine, 2′-O-methyl uridine (Um), 5,2′-O-dimethyl uridine (m⁵Um), 2′-O-methyl pseudouridine (yn), 2-thio-2′-O-methyl uridine (s2Um), 5-methoxycarbonylmethyl-2′-O-methyl uridine (mcm⁵Um), 5-carbamoylmethyl-2′-O-methyl uridine (ncm⁵Um), 5-carboxymethylaminomethyl-2′-O-methyl uridine (cmnm⁵Um), 3,2′-O-dimethyl uridine (m³Um), 5-(isopentenylaminomethyl)-2′-O-methyl uridine (inm⁵Um), 1-thio uridine, deoxythymidine, 2′-F-ara uridine, 2′-F uridine, 2′-OH-ara uridine, 5-(2-carbomethoxyvinyl) uridine, and 5-3-(1-E-propenylamino) uridine.

In some embodiments, the modRNA comprises a modified cytosine selected from the group consisting of 5-aza cytidine, 6-aza cytidine, pseudoisocytidine, 3-methyl cytidine (m³C), N⁴-acetyl cytidine (act), 5-formyl cytidine (f⁵C), N⁴-methyl cytidine (m⁴C), 5-methyl cytidine (m⁵C), 5-halo cytidine (e.g., 5-iodo cytidine), 5-hydroxymethyl cytidine (hm⁵C), 1-methyl pseudoisocytidine, pyrrolo-cytidine, pyrrolo-pseudoisocytidine, 2-thio cytidine (s2C), 2-thio-5-methyl cytidine, 4-thio pseudoisocytidine, 4-thio-1-methyl pseudoisocytidine, 4-thio-1-methyl-1-deaza pseudoisocytidine, 1-methyl-1-deaza pseudoisocytidine, zebularine, 5-aza zebularine, 5-methyl zebularine, 5-aza-2-thio zebularine, 2-thio zebularine, 2-methoxy cytidine, 2-methoxy-5-methyl cytidine, 4-methoxy pseudoisocytidine, 4-methoxy-1-methyl pseudoisocytidine, lysidine (k²C), alpha-thio cytidine, 2′-O-methyl cytidine (Cm), 5,2′-O-dimethyl cytidine (m⁵Cm), N⁴-acetyl-2′-O-methyl cytidine (ac⁴Cm), N⁴,2′-O-dimethyl cytidine (m⁴Cm), 5-formyl-2′-O-methyl cytidine (f⁵Cm), N⁴,N⁴,2′-O-trimethyl cytidine (m⁴₂Cm), 1-thio cytidine, 2′-F-ara cytidine, 2′-F cytidine, and 2′-OH-ara cytidine.

In some embodiments, the modRNA comprises a modified adenine selected from the group consisting of 2-amino purine, 2,6-diamino purine, 2-amino-6-halo purine (e.g., 2-amino-6-chloro purine), 6-halo purine (e.g., 6-chloro purine), 2-amino-6-methyl purine, 8-azido adenosine, 7-deaza adenine, 7-deaza-8-aza adenine, 7-deaza-2-amino purine, 7-deaza-8-aza-2-amino purine, 7-deaza-2,6-diamino purine, 7-deaza-8-aza-2,6-diamino purine, 1-methyl adenosine (m¹A), 2-methyl adenine (m²A), N⁶-methyl adenosine (m⁶A), 2-methylthio-N⁶-methyl adenosine (ms²m⁶A), N⁶-isopentenyl adenosine (i⁶A), 2-methylthio-N⁶-isopentenyl adenosine (ms²i⁶A), N⁶-(cis-hydroxyisopentenyl) adenosine (io⁶A), 2-methylthio-N⁶-(cis-hydroxyisopentenyl) adenosine (ms²io⁶A), N⁶-glycinylcarbamoyl adenosine (g⁶A), N⁶-threonylcarbamoyl adenosine (t⁶A), N⁶-methyl-N⁶-threonylcarbamoyl adenosine (m⁶t⁶A), 2-methylthio-N⁶-threonylcarbamoyl adenosine (ms²g⁶A), N⁶,N⁶-dimethyl adenosine (m⁶₂A), N⁶-hydroxynorvalylcarbamoyl adenosine (hn⁶A), 2-methylthio-N⁶-hydroxynorvalylcarbamoyl adenosine (ms²hn⁶A), N⁶-acetyl adenosine (ac⁶A), 7-methyl adenine, 2-methylthio adenine, 2-methoxy adenine, alpha-thio adenosine, 2′-O-methyl adenosine (Am), N⁶,2′-O-dimethyl adenosine (m⁶Am) N⁶,N⁶,2′-O-trimethyl adenosine (m⁶2Am), 1,2′-O-dimethyl adenosine (m¹Am), 2′-O-ribosyl adenosine (phosphate) (Ar(p)), 2-amino-N⁶-methyl purine, 1-thio adenosine, 8-azido adenosine, 2′-F-ara adenosine, 2′-F adenosine, 2′-OH-ara adenosine, and N⁶-(19-amino-pentaoxanonadecyl) adenosine.

In some embodiments, the modRNA comprises a modified guanine selected from the group consisting of inosine (I), 1-methyl inosine (m¹I), wyosine (imG), methylwyosine (mimG), 4-demethyl wyosine (imG-14), isowyosine (imG2), wybutosine (yW), peroxywybutosine (o₂yW), hydroxywybutosine (OHyW), undermodified hydroxywybutosine (OHyWy), 7-deaza guanosine, queuosine (Q), epoxyqueuosine (oQ), galactosyl queuosine (galQ), mannosyl queuosine (manQ), 7-cyano-7-deaza guanosine (preQ₀), 7-aminomethyl-7-deaza guanosine (preQ₁), archaeosine (G⁺), 7-deaza-8-aza guanosine, 6-thio guanosine, 6-thio-7-deaza guanosine, 6-thio-7-deaza-8-aza guanosine, 7-methyl guanosine (m⁷G), 6-thio-7-methyl guanosine, 7-methyl inosine, 6-methoxy guanosine, 1-methyl guanosine (m¹G), N²-methyl-guanosine (m²G), N²,N²-dimethyl guanosine (m²₂G), N^{2,7}-dimethyl guanosine (m^{2,7}G), N², N²,7-dimethyl guanosine (m^{2,2,7}G), 8-oxo guanosine, 7-methyl-8-oxo guanosine, 1-methio guanosine, N²-methyl-6-thio guanosine, N²,N²-dimethyl-6-thio guanosine, alpha-thio guanosine, 2′-O-methyl guanosine (Gm), N²-methyl-2′-O-methyl guanosine (m²Gm), N²,N²-dimethyl-2′-O-methyl guanosine (m²₂Gm), 1-methyl-2′-O-methyl guanosine (m¹Gm), N^{2,7}-dimethyl-2′-O-methyl guanosine (m^{2,7}Gm), 2′-O-methyl inosine (1m), 1,2′-O-dimethyl inosine (m¹Im), 2′-O-ribosyl guanosine (phosphate) (Gr(p)), 1-thio guanosine, O⁶-methyl guanosine, 2′-F-ara guanosine, and 2′-F guanosine.

modRNA may include, for example, a non-natural or modified nucleotide. The non-natural or modified nucleotide may include, for example, a backbone modification, sugar modification, or base modification. The non-natural or modified nucleotide may include, for example, a base modification. In some embodiments, the base modification is selected from the group consisting of 2-amino-6-chloropurine riboside 5′ triphosphate, 2-aminoadenosine 5′ triphosphate, 2-thiocytidine 5′ triphosphate, 2-thiouridine 5′ triphosphate, 4-thiouridine 5′ triphosphate, 5-aminoallylcytidine 5′ triphosphate, 5-aminoallyluridine 5′ triphosphate, 5-bromocytidine 5′ triphosphate, 5-bromouridine 5′ triphosphate, 5-iodocytidine 5′ triphosphate, 5-iodouridine 5′ triphosphate, 5-methylcytidine 5′ triphosphate, 5-methyluridine 5′ triphosphate, 6-azacytidine 5′ triphosphate, 6-azauridine 5′ triphosphate, 6-chloropurine riboside 5′-triphosphate, 7-deazaadenosine 5′ triphosphate, 7-deazaguanosine 5′ triphosphate, 8-azaadenosine 5′ triphosphate, 8-azidoadenosine 5′ triphosphate, benzimidazole riboside 5′ triphosphate, N¹-methyladenosine 5′ triphosphate, N¹-methylguanosine 5′ triphosphate, N⁶-methyladenosine 5′ triphosphate, O⁶-methylguanosine 5′ triphosphate, N¹-methyl-pseudouridine 5′ triphosphate, puromycin 5′-triphosphate, and xanthosine 5′ triphosphate. Thus, according to some embodiments, the modRNA comprises N1-methyl-pseudouridine 5′ triphosphate.

### Viral Vectors

Viral vector is an effective means of gene transfer. Examples of viral vectors suitable for use herein are retroviral vectors, lentiviral vectors, adenoviral vectors, adeno-associated viral vectors, and other vectors that can integrate into a chromosomal location within the host genome and provide stable expression of a gene of interest. Other vectors include episomal vectors, as well as engineered lentivirus vector variants that are non-integrative. A nucleic acid encoding a desired transcription factor can be inserted into a viral vector and packaged in viral particles using methodologies known in the art. The recombinant viruses can then be isolated and incubated with the intended cells to deliver the transcription factor encoding nucleic acid to the cells.

### Lentiviral Vectors

Lentiviral vectors are vehicles for gene delivery that were originally derived from the human immunodeficiency virus type-1 (HIV-1) lentivirus. These vectors are defective for replication, and thus considered relatively safe, but are capable of stably integrating into the genomic DNA of a broad range of dividing and nondividing mammalian cell types. Engineered lentivirus vector variants that are non-integrative can also be used to deliver a nucleic acid encoding a desired transcription factor.

### Adeno-Associated Viral (AAV) Vectors

Adeno-associated viral (AAV) vectors are replication-defective, single-stranded DNA parvoviruses that require a helper Ad for their replication. Site-specific or random AAV vector integration into the host cell genome, in the absence of a helper virus, results in long-term transgene expression.

### Methods of Making Gastric Insulin Secreting (GINS) Organoids

Another aspect of this disclosure is directed to a method of producing human gastric insulin-secreting (GINS) organoids, which comprises: culturing the human GINS cells obtained herein in a GINS medium for a period of time to allow aggregation of the human GINS cells into human GINS organoids, wherein the human GINS organoids are pancreatic islet-like organoids, insulin-secreting and glucose-responsive.

In some embodiments, the period of time is from about 6 days to about 21 days, e.g., 10 days. In some embodiments, the GINS medium is a chemically defined, serum free medium.

### Gastric Insulin-Secreting (GINS) Medium

The Gastric Insulin-Secreting (GINS) medium is a chemically defined, serum free medium used for GINS differentiation. The composition and concentration of the GINS medium is shown in Table 2 below:

TABLE 2

| | | Final | |
|---|---|---|---|
| Component | Full name, vendor (catalog #) | Concentration | |
| Adv | Advanced DMEM/F-12 (Dulbecco's Modified Eagle | | |
| DMEM/F12 | Medium/Ham's F-12), Gibco (#12634028) | | |
| B27 | B-27 Supplement (50X), Gibco (#17504001) | 1X | |
| N2 | N-2 Supplement (100X), Gibco (#17502048) | 1X | |
| HEPES | HEPES, Gibco (#15630080) | 10 | mM |
| GlutaMAX | 200 mM L-alanyl-L-glutamine dipeptide in 0.85% NaCl, | 1X | (2 mM) |
| | Gibco (#35050079) | | |
| Nicotinamide | 3-pyridinecarboxamide, Sigma-Aldrich (#N5535) | 10 | mM |
| Primocin | Invivogen (#ant-pm-1) (antibiotics, not important for | 25-50 | μM |
| | differentiation) | | |
| NAC | N-acetyl-L-cysteine, Sigma-Aldrich (#A9165) | 0.5-1 | mM |
| Y-27632 | trans-4-[(1R)-1-aminoethyl]-N-4-pyridinyl- | 1-20 | μM |
| | Cyclohexanecarboxamide, dihydrochloride, LC | | |
| | Laboratories, (#Y-5301) | | |
| A8301 | 3-(6-methyl-2-pyridinyl)-N-phenyl-4-(4-quinolinyl)-1H- | 0.5-2 | μM |
| | pyrazole-1-carbothioamide, Cayman, (#9001799) | | |

### Gastric Gene Signature

Gene signatures are important to represent the molecular changes in the disease genomes or the cells in specific conditions and have been often used to separate samples into different groups for better research or clinical treatment. In some embodiments, the gene signatures are used to identify specific cell types, i.e., the specific combinations of genes represent the unique transcriptional identities of cell types. In some embodiments, a gastric score can be calculated based on the expression levels of gastric-specific genes, e.g., using a published statistical method (Tirosh et al, 2016, Science, April 8; 352(6282):189-96). The score card of gastric cells used in the experiment, contain 868 stomach-specific genes, i.e., the gastric gene signature. A smaller representative group of gastric genes include: AGR2, BACE2, CLDN18, DDX21, FABP5, LGALS3, MUC1, RASSF6, SPINK1, and TMEM97. In some embodiments, the gastric score is based on expression levels of some of the 868 stomach-specific genes listed in Table 1, e.g., 50, 100, 200, 300, 400, 500, 600, 700, or 800 genes. In some embodiments, the gastric score is based on expression levels of all of the 868 stomach-specific genes listed in Table 1.

In some embodiments, the term ‘residual gastric gene expression’ represents the average expression values of a cohort of gastric genes. In some embodiments, the residual gastric gene expression is determined by single cell RNA sequencing. The average expression value of the gastric genes (i.e., gastric score) of human GINS cells is statistically higher than that of primary human beta cells (pancreatic 3-cells) but significantly lower than that of bona fide human gastric cells. The human GINS cells thus are considered to retain residual gastric gene expression. The ‘gastric-specific’ expression as disclosed herein is relative to pancreatic islets only. The ‘gastric-specific’ genes are often expressed in multiple tissues in addition to gastric tissue.

### Methods of Controlling Glycemia

GINS cells and GINS organoids can be used in transplantation therapies to control glycemia. Examples of therapy include:
(a) autologous and allogenic cell therapy for T1D (Type 1 Diabetes).
(b) autologous and allogenic cell therapy for T2D (Type 2 Diabetes).
(c) autologous and allogenic cell therapy for patients with partial or complete pancreatectomy.

Either autologous or allogenic cells or organoids can be used. The transplantation site(s) may include liver, muscle, subcutaneous space, fat depot, omentum membrane, abdominal cavity, and others. GINS cells and organoids may be mixed, prior or during transplantation, with other cells including mesenchymal cells, vascular cells, and immune cells (such Treg cells). GINS cells and organoids may be mixed, prior or during transplantation, with compounds, growth factors, mRNA and other chemical, protein, and bio or synthetic materials.

In another aspect, a method of controlling glycemia in a human subject is provided and comprises transplanting to the human subject the population of GINS cells or the preparation of GINS organoids.

In some embodiments, the population of human GINS cells or the preparation of human GINS organoids are transplanted in the liver, muscle(s), a subcutaneous space, a fat depot, an omentum membrane, or an abdominal cavity of the subject. In some embodiments, the population of human GINS cells or the preparation of human GINS organoids are autologous or allogenic relative to the subject. In some embodiments, the human subject is a human subject having type 1 diabetes, type 2 diabetes, or having a partial or complete pancreatectomy.

In some embodiments, the population of human GINS cells or the preparation of human GINS organoids are mixed, prior or during transplantation, with other cells including mesenchymal cells, vascular cells, or immune cells. In some embodiments, the population of human GINS cells or the preparation of human GINS organoids are mixed, prior or during transplantation, with compounds, growth factors, mRNA, other chemical, protein, and bio or synthetic materials. In some embodiments, the population of human GINS cells or the preparation of human GINS organoids are encapsulated or seeded into a device prior or during transplantation.

In another aspect, a method of treating diabetes in a human subject is provided, which comprises transplanting to the human subject a mixture of the population of human GINS cells and the preparation of human GINS organoids.

### Examples

The present description is further illustrated by the following examples, which should not be construed as limiting in any way. The contents of all cited references (including literature references, issued patents, and published patent applications as cited throughout this application) are hereby expressly incorporated by reference.

### Example 1: Materials and Methods

### Lentivirus Packaging and Titration

The lentiviruses were packaged using Lipofectamine 3000 (Thermo Fisher Scientific, L3000001) as previously described (Gu et, al., 2022, STAR Protoc 3, 101308). Viral supernatant was cleared by centrifugation followed by filtration using a 0.45 pm polyethersulfone (PES) filter. Viruses were then concentrated 20-fold by Centrifugal Filter (Millipore Sigma, UFC910024). Concentrated viruses were aliquoted and stored at −80° C. To measure the titer of lentiviruses, HEK293FT (ThermoFisher, R70007) cells were seeded in 24-well plates to be ˜90% confluent at infection. Virus was diluted in DMEM medium containing 10% FBS and 10 g/mL polybrene and added to the cells. Forty-eight hours post infection, the number of fluorescence-positive cells was counted under a fluorescence microscope if the lentiviral vector carried a fluorescence marker (e.g., mCherry for Lenti-EF1a-Ngn3ER-Puro^{R}-mCherry).

Otherwise, transduced cells were visualized by an immunofluorescence assay (e.g., MAFA staining for Lenti-TetO-PM or Lenti-CMV-PM). Viral titers were defined by the transduction units per mL (TU/mL).

### Human Samples

One human biopsy sample (Donor-CH) was used from Boston Children's Hospital under the protocol (IRB-P00000529). All studies involving human samples were approved by ethics committees at Boston Children's Hospital and Weill Cornell Medical College. The autopsy samples were from either the International Institute for the Advancement of Medicine (IIAM) or The National Disease Research Interchange (NDRI). Informed consent was obtained from the participants and or parents/guardian for these studies. Subject details are described in Table 3. Human islets were obtained from Prodo Labs.

TABLE 3
Information of stomach tissue donors

| Donor | Age | | Sex |
|---|---|---|---|
| #6 | 23 | years | Male |
| #7 | 29 | years | Female |
| #9 | new born | | Female |
| #10 | 5 | years | Male |
| CH | 5 | years | Male |

Derivation and Culture of Human Gastric Stem Cells from Primary Mucosal Tissues

Primary human gastric stem cells were isolated as described in detail (Huang et. al., 2023, ProtocolExchange). Human gastric samples (3-5 mm in size) were vigorously washed with cold DPBS 3 times and cut into smaller pieces with a sharp scalpel. Tissue was then incubated with F12K medium containing 2 mg/ml collagenase type IV (Worthington, LS004188) at 37° C. with pipetting every 5-10 min until most of the glandular cells were released and appeared in solution as clusters. Cells were neutralized with F12K supplemented with 10% FBS and centrifuged at 500×g for 5 min. Pelleted cells were resuspended in human gastric stem cell culture medium (hGSC medium) and seeded on mitomycin-C-inactivated mouse embryonic fibroblasts (MEF, E13.5-14.5, DR4 strain, The Jackson Laboratory, 003208) coated 3-cm dish.

The hGSC medium was formulated using a method slightly modified from a published report (Wang et.al., 2015, Nature 522, 173-178): basal medium composed of 66.7% DMEM (high glucose, Thermo Fisher Scientific, 11-965-118) and 33.3% F12K (Thermo Fisher Scientific, 21127030) was supplemented with 18% FBS (R&D systems, S11150), 10% R-Spondin-2 conditioned medium, 10 mM Nicotinamide (Sigma-Aldrich, N5535), 25 μM Primocin (Invivogen, ant-pm-1), 1 μM A8301 (Cayman, 9001799), 5 μg/mL insulin (Sigma-Aldrich, I0516-5ML), 10 μM Y-27632 (LC Laboratories, Y-5301), 1 μM DMH1 (Cayman, 16679), 50 ng/mL EGF (R&D Systems, 236-EG-01M), and 2 μM T3 (Sigma-Aldrich, T6397).

hGSCs were maintained at 37° C. in a 7.5% CO₂ incubator. Culture medium was changed every 2-3 days. Y-27632 was withdrawn 24 h post passage. hGSC colonies were split every 4-6 days at a ratio between 1:3 and 1:5 as follows: cells were washed twice with DPBS and dissociated by 10-12 min incubation in TrypLE (Thermo Fisher Scientific, 12604021) with pipetting at the end. Cells were then neutralized by DMEM medium with 10% FBS and centrifuged at 300×g for 5 min. Pelleted cells were resuspended in hGSC medium and then seeded on an inactivated-MEF-coated dish.

hGSCs Engineering with Lentiviral Infection for Transgene and Reporter Gene Expression

Human gastric stem cells were engineered as described in detail (Huang et. al., 2023, ProtocolExchange). To engineer hGSCs, cells were passaged in one well of 6-well plate 24 hours prior to lentiviral transduction. Cells were washed with DPBS and overlaid with hGSC medium containing 10 g/mL polybrene and 25 μL of lentivirus (viral titer: ˜108 TU/mL). Spinfection was then performed as follows: the cell culture with lentivirus was spun at 1000×g for 30 min at 37° C. and then incubated at 37° C. in a 7.5% CO₂ incubator for 48 hours. The medium was changed to hGSC medium containing 1 g/mL puromycin or 10 g/mL Blasticidin according to the selection marker incorporated into the construct for 2 weeks. The Ngn3ER-hGSCs were labeled with constitutive mCherry expression by incorporation of a polycistronic cassette EF1a-Ngn3ER-Puro^{R}-mCherry (Puro^{R}, puromycin resistant gene). To establish the Ngn3 and Pdx1-Mafa dual inducible cell line (Ngn3ER/TetOPM-hGSCs), Ngn3ER-hGSCs were infected by Lenti-TetO-PM (lentivirus carrying the polycistronic cassette TetO-Pdx1-Mafa) and PGK-rtTA-Blast^{R} (Blast^{R}, blasticidin resistant gene). To establish the pGAL-GFP reporter cell line (Ngn3ER/pGAL-GFP-hGSCs), Ngn3ER-hGSCs were infected by lentivirus carrying transgenic GAL promoter (1,591 bp, upstream region of the TSS (transcription start site)) driven GFP reporter (pGAL-GFP) and PGK-Blast^{R}. Doxycycline (Dox, 1 g/mL) or 4-OH-Tam (1 μM, Sigma-Aldrich, H7904) were added to the medium to induce the expression of TetO-driven transcription or Ngn3ER activation.

### Supplement Screen to Formulate Chemically Defined Serum-Free Medium for GINS Differentiation.

Ngn3ER/PM-hGSCs were seeded 5 days prior to differentiation in 96-well plate. To start differentiation, 1 μM 4-OH-TAM was added and incubated for 2 days. The culture medium was then changed to serum-free basal medium with one supplement and Doxycycline to activate Pdx1-Mafa expression. The basal serum-free medium for screens was prepared as follows: advanced DMEM/F12 (Thermo Fisher Scientific, 12634010) was supplemented with 10 mM HEPES (Thermo Fisher Scientific, 15-630-080), 1× GlutaMAX (Thermo Fisher Scientific, 35050061), 1× B-27 (Themo Fisher Scientific, 17504044), 1× N-2 (Thermo Fisher Scientific, 17502048), 25 μM Primocin, and 500 μM N-Acetyl-L-Cysteine (NAC) (Sigma-Aldrich, A9165). Seven days after supplement treatment, the following were (1) spontaneous clustering of nascent GINS cells by live imaging of mCherry⁺ cells, and (2) qPCR of INS mRNA levels. Then multiple b-cell markers with qPCR on samples treated with Nicotinamide, Y-27632 and A8301 were analyzed.

### Generation of GINS Organoids

Mouse ECs GINS organoids were generated as described in detail (Huang et. al., 2023, ProtocolExchange). Plasmids were available from Addgene under a uniform biological material transfer agreement. The procedure was briefly summarized as follow:

(1) Ngn3ER activation (Differentiation to endocrine progenitors) (day 0-2): Ngn3ER-hGSCs were seeded 4-5 days prior to differentiation. Cells were washed with DPBS and overlaid with hGSC medium containing 1 μM 4-OH-TAM.

(2) Pdx1-Mafa transduction (Differentiation to GINS precursors) (day 2-6): Endocrine progenitors were gently washed with DPBS, incubated in DPBS for 5-10 min, and detached by pipetting. Pelleted cells were then digested in TrypLE at 37° C. for 10 min with pipetting every 3-5 min. Dissociated endocrine progenitors were transduced by Lenti-CMV-PM (lentivirus carrying the polycistronic cassette CMV-Pdx1-Mafa) at an MOI of 10 by spinfection in medium composed of 50% of hGSC medium, 50% of GINS medium, and 10 μg/mL polybrene. Cells were then transferred to tissue culture dishes (˜10⁷ cells per 10-cm dish) coated with Fibronectin (1:50, Sigma-Aldrich, F4759) and Matrigel (1:50, VWR, 47743-722). On day 4, the culture medium was changed to medium consisting of 75% GINS medium and 25% hGSC medium.

(3) GINS organoid formation (day 6-21): GINS precursors were dissociated by 5-10 min TrypLE treatment and aggregated (typically 2.0-2.4 million cells/well) in AggreWell400 (STEMCELL Technologies, 34450) using the manufacturer's recommended protocol. Medium was changed every 2-3 days. Aggregates normally formed within 24 hours.

(4) GINS medium for this study was formulated as the follows: advanced DMEM/F12 supplemented with 10 mM HEPES, 1× GlutaMAX, 25 μM Primocin, 500 μM NAC, 1× B-27, 1× N-2, 10 mM Nicotinamide, 1 μM A8301, and 10 μM Y-27632.

Alternatively, the dual inducible cell line Ngn3ER/TetOPM-hGSCs was used for differentiation optimization (Table 4). Instead of lentiviral transduction at step (2), 1 g/mL Dox was added to the medium starting on day 2 to induce expression of Pdx1-Mafa.

TABLE 4
Differentiation protocol of GINS organoids

| | Duration | | |
|---|---|---|---|
| Stage | (days) | Medium | Step |
| Endocrinc | 2 | hGSC mcdium¹ | Addition of 1 μm |
| progenitors | | | 4-OH-TAM |
| GINS | 2 | 50% hGSC medium | Infection of Lenti- |
| precursors | | 50% GINS medium² | CMV-PM (MOI 10) |
| | 2 | 25% hGSC medium | Medium change |
| | | 75% GINS medium | |
| GINS | 4-18 | GINS medium | Aggregation and |
| organoids | | | culture |

¹hGSC medium: basal = 66.7% DMEM and 33.3% F12K; supplements = 18% FBS, 10% R-Spondin-2 conditioned medium, 10 mM Nicotinamide, 1 μM A8301, 5 μg/mL insulin, 10 μM Y-27632, 1 μM DMH1, 50 ng/mL EGF, 2 μM T3.
²GINS medium: basal = advanced DMEM; supplements = 10 mM HEPES, 1X GlutaMAX, 1X B-27, 1X N-2, 500 μM N-Acetyl-L-Cysteine, 10 mM Nicotinamide, 1 μM A8301, and 10 μM Y-27632.

### Static Glucose-Stimulated Insulin Secretion (GSIS)

Ten to twenty GINS organoids were sampled for each group. Organoids were washed with RPMI-1640 no nutrient medium (RPMIN, MyBioSource, MBS652918), and equilibrated in 3.3 mM glucose RPMIN for 2 hours. Organoids were incubated in low-glucose RPMIN for 1 hour, and supernatant was collected. Organoids were then incubated in high-glucose RPMIN for 1 hour, and supernatant was collected. For sequential glucose challenge, organoids were washed 2 times in RPMIN prior to the next stimulation. Secreted insulin was measured using the Stellux Chemi Human Insulin ELISA (ALPCO Diagnostics, 80-INSHU-CH10).

Dynamic GSIS with Perifusion

Twenty GINS organoids were sampled for each group. Organoids were washed with RPMIN and equilibrated in 1 mM glucose RPMIN for 2 hours at 37° C. incubator. The assay was performed in RPMIN on a temperature-controlled (37° C.) perifusion system (Biorep Technologies). Organoids were loaded in chambers and perfused at a flow rate of 100 μL/min in the following steps: (1) 48 min in 1 mM glucose, (2) 24 min in 2 mM glucose. (3) 36 min in 20 mM glucose, (4) 24 min in 20 mM glucose with 100 ng/mL Liraglutide (Cayman, 24727), (5) 48 min in 2 mM glucose, (6) 20 min in 2 mM glucose with 30 mM KCl. Secreted insulin was measured using the Stellux Chemi Human Insulin ELISA.

### Transplantation Studies in Normoglycemic and Hyperglycemic Mice

All mouse experiments were conducted under the IACUC protocol 2018-0050 at Weill Cornell Medical College (WCMC). Mice were housed in a temperature- and humidity-controlled environment with 12 hours light/dark cycle and food/water ad libitum. Mice were fed with chow diet (PicoLab Rodent Diet 5053). NSG mouse breeders were purchase from the Jackson Laboratory (Strain #:005557). NSG breeding was conducted in WCMC. Transplantations were performed with 8-12 weeks old male NSG mice. GINS organoids or human islets were transplanted under the capsule of the left kidney in NSG mice anesthetized with isoflurane. For glucose tolerance test, transplanted mice were fasted for 6 hours and injected with 2 g/kg glucose intraperitoneally. Blood glucose was then measured at indicated time points by glucometer. For in vivo GSIS, transplanted mice were fasted overnight and injected with 2 g/kg glucose intraperitoneally. Blood before injection and 1 hour post glucose injection was collected by submandibular bleeding with Microvette 300 capillary blood collection tube (Sarstedt, 20.1309.100). Serum was separated from the blood for insulin measurement by Stellux Chemi Human Insulin ELISA. For the diabetes rescue experiment, male mice were injected with four doses of Streptozotocin (35 mg/kg/d) on four consecutive days to induce hyperglycemia. Mice that showed hyperglycemia (>250 mg/dl) on four consecutive days were selected for transplantation. Sham transplantation was conducted by operating the surgical procedure without infusing cells. Random fed blood glucose was monitored 2 times per week. To remove grafts, a survival nephrectomy was performed after 90-100 days post transplantation. Briefly, the left kidney was ligated at the renal hilum using 3-0 silk and then resected.

### Immunofluorescence

GINS organoids were fixed in 4% PFA for 15 min at room temperature. Kidney samples were fixed in 4% PFA at 4° C. for 1 hour. Samples were washed in PBS, incubated in PBS containing 30% sucrose overnight. Samples were frozen in OCT (Tissue-Tek) next day, and then cryo-sectioned. Following PBS wash, sections were blocked for 1 hour at room temperature in blocking buffer: 10% normal donkey serum (Jackson ImmunoResearch, 017-000-121) in PBST (0.1% TritonX-100 in PBS). Sections were then incubated with primary antibodies in blocking buffer overnight at 4° C. The following primary antibodies were used in this study: at anti-C-peptide (DHSB; GN-ID4; 1:300), rabbit anti-somatostatin (Dako; A0566, 1:500), goat anti-ghrelin (Santa Cruz, sc-10368, 1:400), guinea pig anti-glucagon (Linco, 4031-01F, 1:2000), rabbit anti-MAFA (Bethyl; A700-067; 1:1000), guinea pig anti-insulin (Dako; A0564; 1:2000), rat anti-CD31 (BD, 550274, 1:50), mouse anti-ENTPD3 (NTPDase3) (developed in house, available at ectonucleotidases-ab.com, 1:1000), rabbit anti-PAX6 (Millipore; AB2237, 1:1000), mouse anti-NKX2-2 (DSHB; 74.5A5-s, 1:25), rabbit anti-PCSK1 (Millipore, AB10553, 1:500), mouse anti-GAL (Santa Cruz, sc-166431, 1:2000), rabbit anti-SOX9 (Santa Cruz, sc-20095, 1:50), mouse anti-K167 (BD, 556003. 1:500). Slides were washed three times in PBST, followed by secondary antibody incubation in blocking buffer with DAPI for 1 hour at room temperature (protected from light). Following 3 washes in PBST, slides were mounted in mounting medium (Vector Laboratories, H-1700-10) and covered with coverslips. The representative images were captured using either a confocal microscope (710 μMeta) or a Nikon fluorescence microscope. Images were processed by Zen (3.4 blue edition) or ImageJ (1.53t).

### RNA Extraction, Reverse Transcription, and Real-Time PCR

RNA was extracted (Qiagen, 74034) and reversely transcribed (Thermo Fisher Scientific, 43-688-13) to complementary DNA (cDNA). cDNA was diluted and then quantified by real-time PCR with TaqMan assay listed in Table 5. For optimization experiments, Cells-to-Ct Kit was used (Thermo Fisher Scientific, A35377).

TABLE 5
Taqman assay list for qPCR

| Gene Target | Assay Number |
|---|---|
| ABCC8 | Hs01093752_m1 |
| ACTB | Hs01060665_g1 |
| G6PC2 | Hs01549773_m1 |
| GCG | Hs01031536_m1 |
| GCK | Hs01564555_m1 |
| GHRL | Hs01074053_m1 |
| INS | Hs00355773_m1 |
| INSM1 | Hs00357871_s1 |
| ISL1 | Hs00158126_m1 |
| KCNJ11 | Hs00265026_s1 |
| NKX2-2 | Hs00159616_m1 |
| NKX6-1 | Hs00232355_m1 |
| RFX6 | Hs00941591_m1 |
| PAX4 | Hs00173014_m1 |
| PAX6 | Hs01088114_m1 |
| PDX1 | Hs00236830_m1 |
| SLC30A8 | Hs00545183_m1 |
| SST | Hs00356144_m1 |

### Fluorescence-Activated Cell Sorting (FACS)

For quantitative flow cytometry, GINS organoids were dissociated in TrypLE for 40 min. Dissociated cells were stained with Fixable Viability Dye 455UV (Thermo Scientific, 65-0868-14) according to the manufacturer's manual. Cells were then fixed and permeabilized using Intracellular Fixation & Permeabilization Buffer Set (Thermo Scientific, 88-8824-00) according to the manufacturer's manual. Fixed cells were then incubated in 1× permeabilization buffer with primary antibodies for 1h at room temperature and washed with permeabilization buffer for 3 times and resuspended in flow cytometry staining buffer (Thermo Scientific, 00-4222). Stained cells were then passed through a 40 m nylon strainer before being sorted. The following primary antibodies were used in this study: mouse anti-C-peptide Alexa Fluor®647 (BD, 565831, 1:25), mouse anti-glucagon Alexa Fluor®488 (R&D, IC1249G, 1:25), mouse anti-somatostatin Alexa Fluor®488 (BD, 566032, 1:25).

For live cells sorting, cells were dissociated in TrypLE (40 min for hGSCs, 5 min for endocrine progenitors, 5 min for GINS precursors and 40 min for GINS organoids), and pelleted. Cells pellet was resuspended in FACS buffer (1% glucose, 10 mM HEPES, 10 μM Y-27632, 1 mM N-acetyl-1-cysteine, and 2% FBS in DPBS) and passed through a 40 pm nylon strainer before being sorted.

To purify GAL-GFP⁺ cells, Ngn3ER/pGAL-GFP-hGSCs were seeded and differentiated toward GINS cells. On day 7 post differentiation, cells were sorted for GFP⁺ cells. Sorted cells were then aggregated into organoids and analyzed 1 and 14 days post aggregation. FACS data was analyzed by FACS DIVA 8.0.1 or FCS Express 7 (7.16.0035).

Single Cell RNA-Seq (scRNA-Seq) from Cultured and Transplanted GINS Organoids and Multiplex scRNA-Seq.

For the time-course study of GINS generation, samples at different time points were harvested on the same day from parallel cultures. Cells were dissociated in TrypLE. FACS sorting was conducted to purify mCherry⁺ DAPI cells. Purified samples were then multiplexed according to the 10× genomics protocol CG000391. Briefly, 2×10⁵ sorted cells from each time point were washed in PBS with 0.04% BSA (Millipore Sigma, A1595), and then labeled with multiplexing oligo individually for 5 min at room temperature. Following two washes in PBS with 1% BSA, equal number of labeled cells from different time points were then pooled. In total 30,000 cells were then loaded for 10× genomics. To compare corpus and antral GINS organoids, cells were dissociated by TrypLE on day 21 post induction. To harvest GINS cells from the grafts, the grafts under the kidney capsule were removed with a scalpel and minced. The tissues were digested with type III collagenase (300U/ml in RPMI 1640) for 1 hour, followed by 5-10 min TrypLE treatment with pipetting. Digested tissue was filtered through 40 m nylon strains and purified by FACS with mCherry⁺ and DAPT⁻ gating. Samples were kept in GINS medium on ice until ready to be processed by 10× genomics single-cell droplet sample preparation workflow at the Genomics Core Facility at Weill Cornell Medicine as previously described (Gu et.al., 2022, Cell Stem Cell, January 6; 29(1):101-115).

scRNA-seq Analysis

(1) Demultiplexing and reads alignment: Human islets (donor #1, #2, #3, #4 and #9) scRNA-seq datasets were downloaded from Gene Expression Omnibus (GEO) database (GSE114297) (Xin et.al., 2018, Diabetes 67, 1783-1794). Multiplexed sequencing data from the Illumina NovaSeq6000 were demultiplexed using the ‘multi’ pipeline from Cell Ranger (v6.1.2). Each of the four Cell Multiplex Oligo labels was assigned a unique sample ID. All the datasets were then processed with the 10× built mouse and human reference ‘refdata-gex-GRCh38-and-mm10-2020-A’z’.

(2) Quality control and count normalization: Cell Ranger outputs were used as input to create Seurat objects by Seurat (v4.1.1) (Butler et.al., 2018, Nat Biotechnol 36, 411-420). Cells that express more murine genes than human genes were defined as contaminant murine cells (e.g., murine host cells from kidney) and removed from the datasets. Murine features were then removed. Low-quality cells were removed as follows: In general, cells were considered low-quality if the number of detectable genes or read counts is below the 3^{rd} percentile or above the 97⁺ percentile of the datasets, or percentage of mitochondrial genes is more than 18. NormalizeData function was used for normalization with default parameters. Putative doublet cells were identified by DoubletFinder (2.0.3) and removed (McGinnis et.al., 2019, Cell Syst 8, 329-337). SoupX (1.6.1) was used to remove ambient RNA in the human islet datasets (Young et.al., 2020, Gigascience 9).

(3) Scaling, dimension reduction, cell clustering, differential expression analysis and cell annotation: The normalized data was scaled by ScaleData function with mitochondrial genes percentage regressed out. Cell cycle status was inferred by CellCycleScoring function and regressed out for the time course study. Principal component analysis (PCA) was performed on the scaled data by runPCA. To place similar cells together in 2-dimensional space, selected top principal components (PCs) of human islet, GINS organoid (corpus), antral GINS organoid, GINS generation time course and GINS graft (corpus) were used as input respectively in non-linear dimensional reduction techniques including tSNE and UMAP. The same PCs of each sample were used to construct K-nearest neighbor (KNN) graph by FindNeighbors function. To cluster cells, FindClusters function was used with a range of resolution between 0.2 to 2. Cells clustered by different resolutions were all visualized by DimPlot function. To identify markers of each cell cluster, FindAllMarkers function was implemented using the following parameters: only.pos=TRUE, min.pct=0.3, logfc.threshold=0.3. Human islets cells were firstly automatically annotated by the R package SingleR (1.10.0) with a publish islets dataset as reference (Muraro et.al., 2016, Cell Syst 3, 385-394). The annotation of the islet dataset was then slightly modified according to the clustering results and cluster markers. Non-endocrine cells or unclear cell types were removed from the human islet dataset. All the other datasets were annotated manually according to markers and integration results.

(4) Integration analysis: GINS organoids (corpus) were integrated with antral GINS organoids, human islets endocrine cells, and GINS grafted cells respectively. The genes that were used for integration were chosen by SelectIntegrationFeatures function with default parameter. Integration anchors were identified by FindIntegrationAnchors function and used to integrate two datasets together with IntegrateData function. All the integrated datasets were scaled with mitochondrial genes regressed out. Dimensions were reduced by PCA, tSNE and UMAP.

(5) Identity scoring: Signature gene sets (Table 1) were downloaded from cell type signature gene sets (C8 collection) of Molecular Signature Database (v7.5.1). Specifically, the gastric signature is a gene list containing curated cluster markers for gastric chief, immature pit, mature pit, isthmus, neck, and parietal cells identified in the study (Busslinger et.al, 2021, Cell Rep 34, 108819), while the signature of b-cell is the MURARO_PANCREAS_BETA_CELL gene set (Muraro et.al., 2016, Cell Syst 3, 385-394). Both gene sets were then applied as inputs in the AddModuleScore function of Seurat with default parameters, which calculates module scores for feature expression programs on single-cell level.

(6) Pseudotime trajectory, RNA velocity, regulon and Gene Oncology (GO) analysis: Seurat object of the time course dataset was converted to monocle 2 (2.24.0) CellDataSet object (Trapnell et.al., 2014, Nat Biotechnol 32, 381-386; Qiu et.al., 2017, Nat Methods 14, 309-315; Qiu et.al., 2017, Nat Methods 14, 979-982). Size factor and dispersion were estimated by estimateSizeFactors and estimateDispersions function respectively. Cell type markers identified by Seurat were sorted based on adjusted p-value. Top 100 markers of each cell type were marked by setOrderingFilter function for later trajectory construction. Data dimension was then reduced by reduceDimsion function with the following arguments: max_components=2, method=‘DDRTree’). Cells were then ordered along the trajectory by orderCells function. To build pseudotime trajectory on UMAP, Seurat object was converted to monocle 3 (1.2.9) object by as.cell_data_set function. Cells were then clustered by cluster_cells function. The trajectory of GINS precursor, δ-like, β-like and α-like cells, which were in the same partition, was constructed by learn_graph followed by order_cells function. RNA velocity was evaluated by RunVelocity function provided by R packages velocyto.R (0.6) and SeuratWrappers (0.3.0) (La Manno et.al., 2018, Nature 560, 494-498). Regulon activity was computed by SCENIC (1.3.1) R package as previously described (Aibar et.al., 2017, Nat Methods 14, 1083-1086). GO analysis was done by enrichGO function in ClusterProfiler (4.4.4) as previously described (Yu et.al., 2012, OMICS 16, 284-287; Wu et.al., 2021, Innovation (N Y) 2, 100141).

### Statistics and Reproducibility

Statistical analysis and figure plotting were done using GraphPad Prism 9 or R (4.2.2). P values are provided in the figures. Sample size, and statistical methods are described in figure legends. Data are presented as the mean±s.d. or s.e.m. as indicated. All experiments were repeated as indicated; n indicates the numbers of independent repeats as indicated. Male mice of similar age were used for transplantation and randomly assigned to experimental groups. No statistical methods were used to pre-determine sample sizes but our sample sizes are similar to those reported in previous publications (Nair et.al., 2019, Nat Cell Biol 21, 263-274; Pagliuca et.al., 2014, Cell 159, 428-439). Data distribution was assumed to be normal but this was not formally tested. Single cells with poor quality sequencing data were excluded as described. For transplantation experiments with diabetic mice, mice that did not develop hyperglycemia after STZ treatment were not used. Data collection and analysis were not performed blind to the conditions of the experiments.

### Data Availability

Sequencing data that support the findings of this study have been deposited in the GEO under accession code GSE205766. Previously published human islet scRNA-seq data (donors #1, #2, #3, #4 and #9) (Xin et.al., 2018, Diabetes 67, 1783-1794) that were reanalyzed in this study are available under accession code GSE114297 (Xin et.al., 2018, Diabetes 67, 1783-1794). Source data are provided with this study. All other data supporting the findings of this study are available from the corresponding author on reasonable request.

### Example 2: Generating Islet-Like Organoids from Human Stomach Samples

Previous study in mice suggested that stomach tissues were more amenable to adopting 3-cell fate than intestinal tissues (Ariyachet et.al., 2016, Cell Stem Cell 18, 410-421). Therefore, this study is focused on using human gastric stem cells (hGSCs) to generate insulin-secreting cells. The human stomach has three distinct parts: the corpus, pylorus (antrum), and cardia, with corpus mucosa being most abundant. Biopsy samples from all three regions have been grown successfully as organoids in three-dimensional (3D) Matrigel or as 2D flat stem cell colonies, while maintaining their regional identity in culture (Wang, et.al., 2015, Nature 522, 173-178; Sato, et.al., 2009, Nature 459, 262-265; Sato, et.al., 2011, Gastroenterology 141, 1762-1772). After in vitro differentiation, hGSCs produce gastric mucosal cells including acid- and mucus-secreting cells (Wang, et.al., 2015, Nature 522, 173-178; Sato, et.al., 2009, Nature 459, 262-265; Sato, et.al., 2011, Gastroenterology 141, 1762-1772). In this study, primarily corpus tissues were used because of its ready availability.

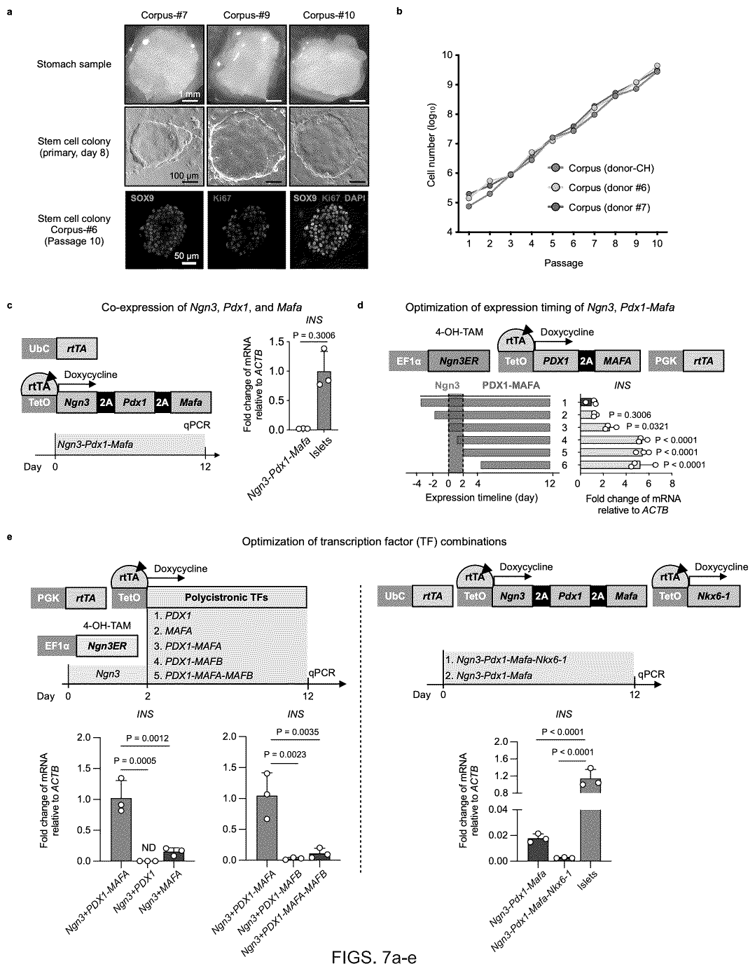

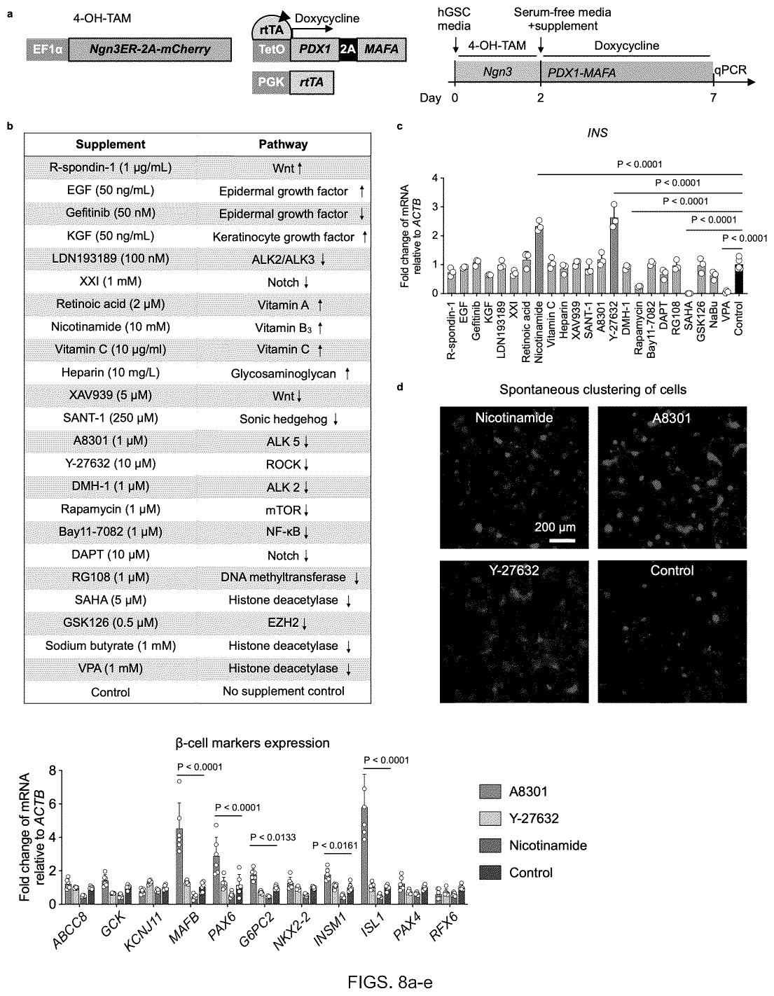

For the ease of scaling stem cell production, 2D culture was used to expand hGSCs. Each biopsy-sized gastric sample typically yielded 30-40 primary colonies, which can be amplified to >10⁹ cells within 2 months (FIGS. 7a, b). Cultured hGSCs continued to express the stomach stem/progenitor marker SOX9 and the proliferative marker KI67 after many passages (FIG. 7a), consistent with prior report (Wang, et.al., 2015, Nature 522, 173-178). To explore ways to direct hGSCs into functional insulin secretors, the study began with the NPM factors (Ngn3, Pdx1, and Mafa). This combination has been shown to confer varying degrees of b-cell properties to non-b cells including pancreatic acinar cells, duct cells, and others (Zhou et al., 2008, Nature 455, 627-632; Hickey et al., 2013, Stem Cell Res 11, 503-515; Li et al., 2014, Nat Biotechnol 32, 1223-1230; Lee et al., 2013, Elife 2, e00940; Furuyama et al., 2019, Nature 567, 43-48). However, co-expression of the NPM factors using the previously published polycistronic cassette yielded low insulin expression in cultured hGSCs (FIG. 7c). Therefore, this study systematically evaluated conditions that may influence GINS cell formation including the timing of NPM expression, inclusion of additional genetic factors, and medium composition. Several notable observations include: (1) high-level insulin induction required transient NGN3 (for 2 days) followed by stable PDX1 and MAFA expression. This sequence of transgene activation was superior to NPM co-expression or expressing PDX1-MAFA prior to NGN3 (FIG. 7d); (2) inclusion of additional b-cell fate regulators such as MAFB or NKX6-1 did not enhance insulin activation (FIG. 7e). Critically, a fully chemically defined serum-free medium was formulated for GINS cell differentiation. From a screen of 23 supplements, some of which are employed in the induction of b-like cells from pluripotent stem cells, it was found that Nicotinamide and Y-27632 (a ROCK inhibitor) significantly promoted INS mRNA levels whereas A8301 (an ALK5 inhibitor) stimulated spontaneous aggregation of nascent GINS cells and expression of several key b-cell transcription factors (FIG. 8). The final GINS differentiation medium contained the three supplements, N2, B27, and N-acetyl cysteine in the basal Advanced DMEM/F12 medium.

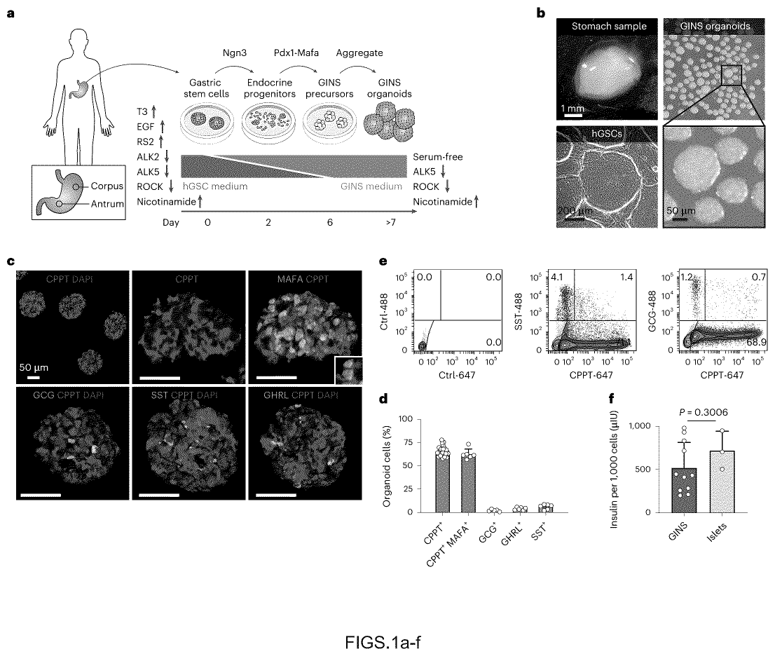

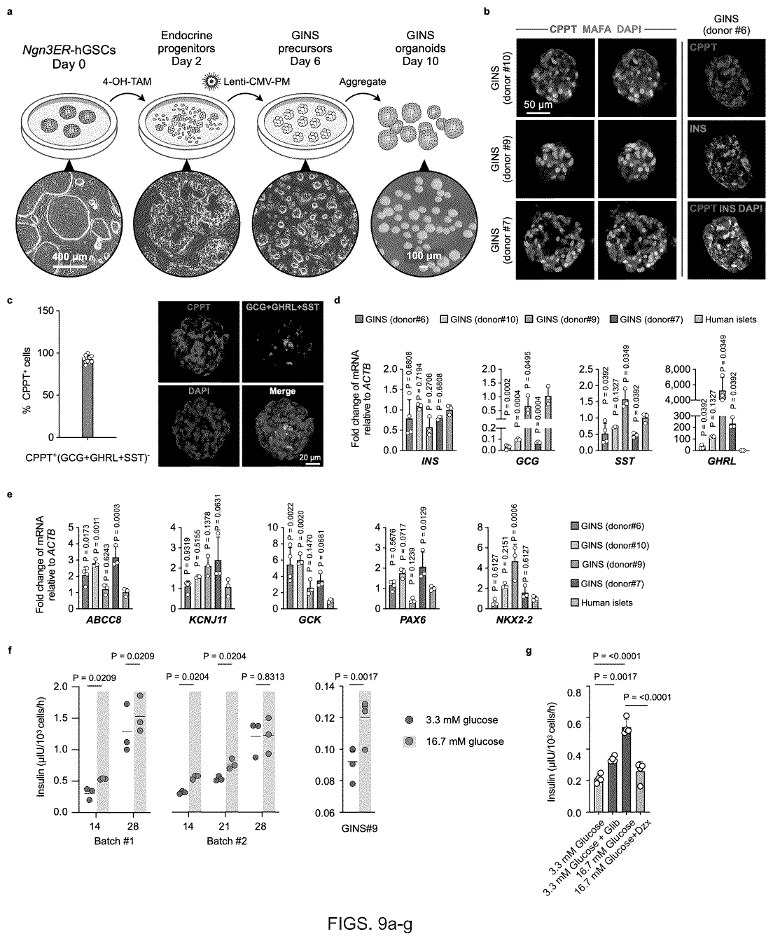

For inducible NGN3 activation, a Ngn3 and estrogen receptor (ER) fusion gene (Ngn3ER) was incorporated into the hGSCs by lentivirus (Johansson et al., 2007, Dev Cell 12, 457-465). hGSC differentiation was initiated by 4OH-Tamoxifen treatment of cultured Ngn3ER-hGSCs for two days (step 1), followed by lentiviral integration of a Pdx1-Mafa co-expression cassette (over 95% infection rate; step 2). Four days later, the nascent GINS cells were aggregated into spherical organoids (step 3), which can persist in the defined medium for up to four weeks (FIGS. 1a, b, FIG. 9a). Immunohistochemistry of GINS organoids from multiple donors revealed that majority of the organoid cells expressed c-peptide (CPPT, 65.4%±5.2%), whereas a minority expressed either glucagon (GCG, 2.2%±1.3%), somatostatin (SST, 6.1%±2.7%), or ghrelin (GHRL, 4.0%±1.5%) (FIGS. 1c, d, FIG. 9b). The cellular composition of organoids was further evaluated by flow cytometry, with the fractions of CPPT⁺, SST⁺ and GCG⁺ cells largely concordant with the immunostaining data (FIG. 1e). Using a cocktail of SST, GCG and GHRL antibodies, it was determined that approximately 92.6% of all CPPT⁺ cells (or 61.0% of all organoid cells) were mono-hormonal (FIG. 9c). The average insulin mRNA levels and insulin content of GINS organoids were comparable to primary islets (FIG. 1f, FIG. 9d). GINS organoids from multiple donors expressed key b-cell markers ABCC8, KCNJ11, GCK, PAX6, and NKX2-2, at comparable levels to primary islets (FIG. 9e).

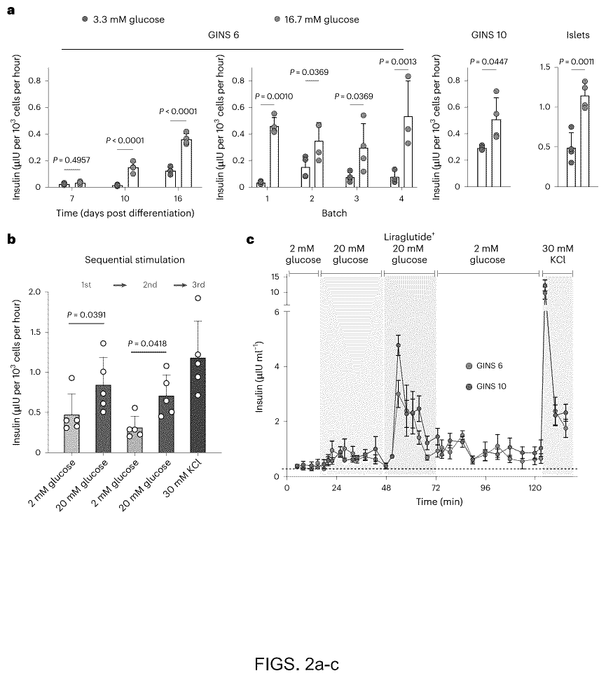

GINS organoids acquired glucose-stimulated insulin secretion (GSIS) 8-10 days after differentiation (FIG. 2a). In contrast, nascent GINS cells had no or little insulin secretion (Day 7, FIG. 2a). Thus, although a small amount of exogenous insulin was present in culture medium, it was not absorbed in any significant way by the cultured cells and insulin engaged with cell surface receptors has been shown to be shuttled intracellularly for degradation (Duckworth et. al., 1998, Endocr Rev 19, 608-624). Notably, glucose responsive organoids could be produced from multiple donors and maintained a batch to batch consistency in functionality (FIG. 2a, FIG. 9f). However, the glucose responsiveness of GINS organoids became less consistent after prolonged culture (over 21 days) (FIG. 9f). GINS organoids responded to repeated glucose challenges as well as the clinical anti-diabetic drug Glibenclamide and the anti-hypoglycemia drug Diazoxide (FIG. 2b, FIG. 9g). In dynamic GSIS assays, GINS cells from two separate donors responded robustly to KCl and liraglutide, a GLP-1 analog, but less so to high glucose challenge (FIG. 2c), indicating that they have not attained full functional maturity. Altogether, these data establish a GINS differentiation protocol robust for different donor tissues, yielding glucose-responsive organoids at high efficiency.

### Example 3: GINS Organoids Contain Four Endocrine Cell Types

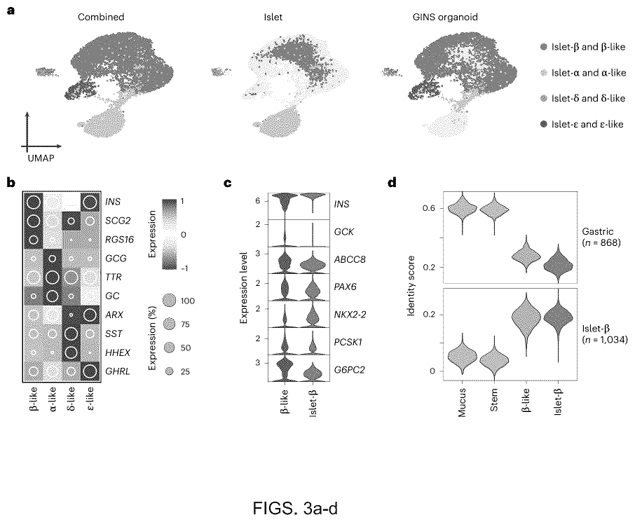

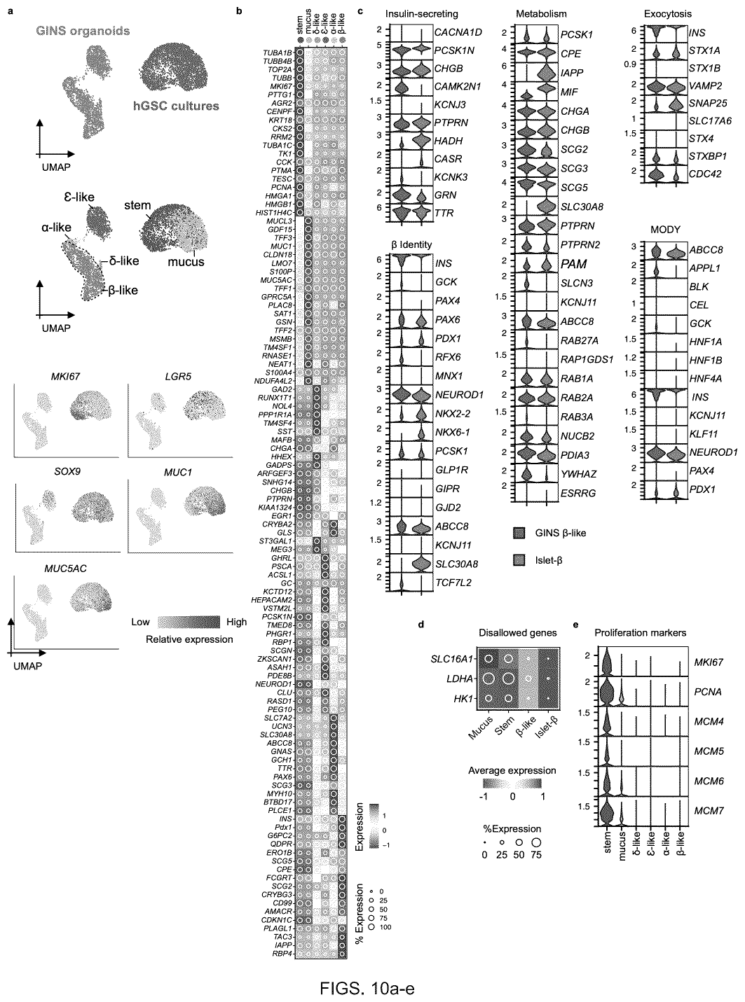

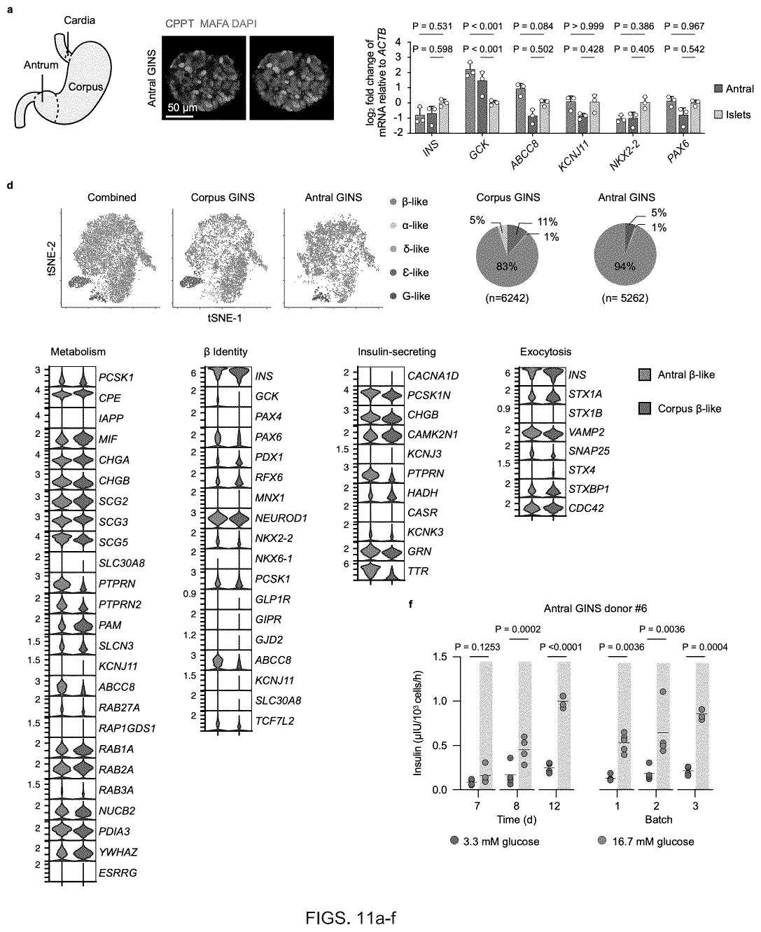

To better understand the identities of the cells in GINS organoids, scRNA-seq was used to interrogate the transcriptomes of 6,242 organoid cells (FIG. 3a). Clustering with published scRNA transcriptomes of human islets revealed four endocrine cell types that aligned with islet β-, α-, δ-, or ε-cells (Xin et.al., 2018, Diabetes 67, 1783-1794) (FIG. 3a). Clustering with hGSCs and mucus-secreting cells (derived from spontaneous hGSC differentiation in culture) showed almost no gastric cells remaining in GINS organoids (FIGS. 10a, 10b). The α- and δ-like endocrine cells expressed canonical markers of their islet counterparts including GCG, ARX, TTR, and GC in ca-cells, and SST and HHEX in δ-cells (FIG. 3b, FIG. 10b). GINS cells expressed classical human β-cell markers including G6PC2, GCK, ABCC8, NKX2-2, PCSK1, PAX6, and key genes involved in β-cell identity, metabolism, insulin synthesis and secretion, and ion channel activities, but did not express NKX6-1 (FIG. 3c, FIG. 10c). Several genes previously shown to interfere with proper glucose sensing, including HK1, LDHA, and SLC16A1 (Pullen et.al., 2010, Islets 2, 89-95) were strongly down-regulated in GINS cells (FIG. 10d). GINS organoid cells ceased proliferation after differentiation (FIG. 10e). Single-cell transcriptomes of antral GINS organoids also revealed a dominant fraction of β-like cells. No significant numbers of α- and ε-like cells were detected whereas an additional gastrin-expressing cell population was present in antral organoids (FIGS. 11a-e). Antral organoids exhibited robust GSIS in vitro (FIG. 11f).

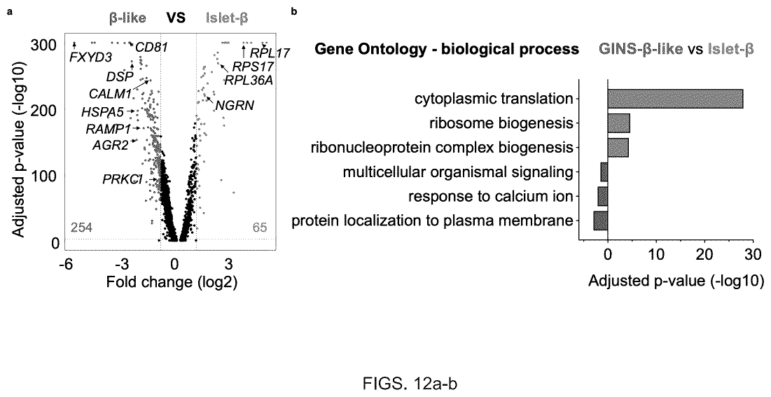

To further assess the identity of GINS cells, molecular score cards of β-cells (1,034 β-cell-specific genes) and gastric cells (868 stomach-specific genes) benchmarked from published human scRNA data (Muraro et.al., 2016, Cell Syst 3, 385-394; Busslinger et.al., 2021, Cell Rep 34, 108819) were applied. GINS cells scored similarly to islet β-cells in both categories, although there was a residual gastric signature in GINS cells (FIG. 3d). In comparison, hGSCs and mucus-secreting cells possessed low β- and high gastric scores (FIG. 3d). These data indicated that GINS cells possessed the general molecular identity of islet β-cells at the single-cell level, consistent with their glucose responsiveness. Nevertheless, Gene Ontology analysis suggested that GINS cells have lower ribonucleoprotein biogenesis activity than islet J-cells (FIG. 12), possibly underlying the functional immaturity of GINS cells in vitro.

### Example 4: GINS Cells Persist after Engraftment and Reverse Diabetes

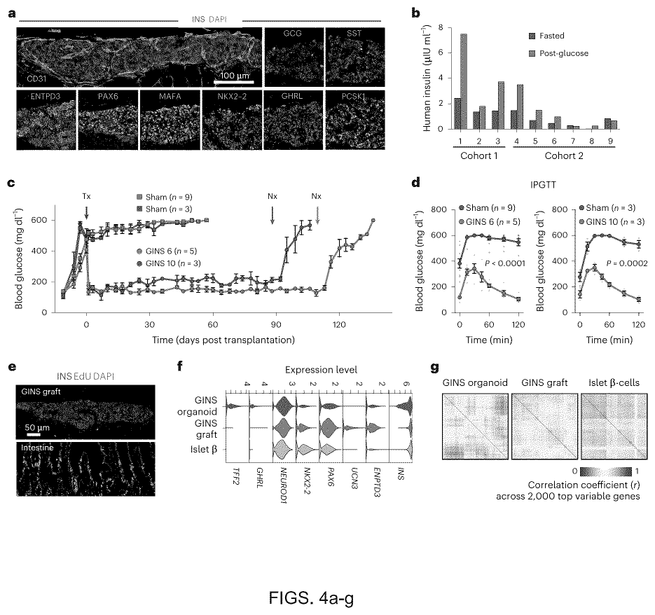

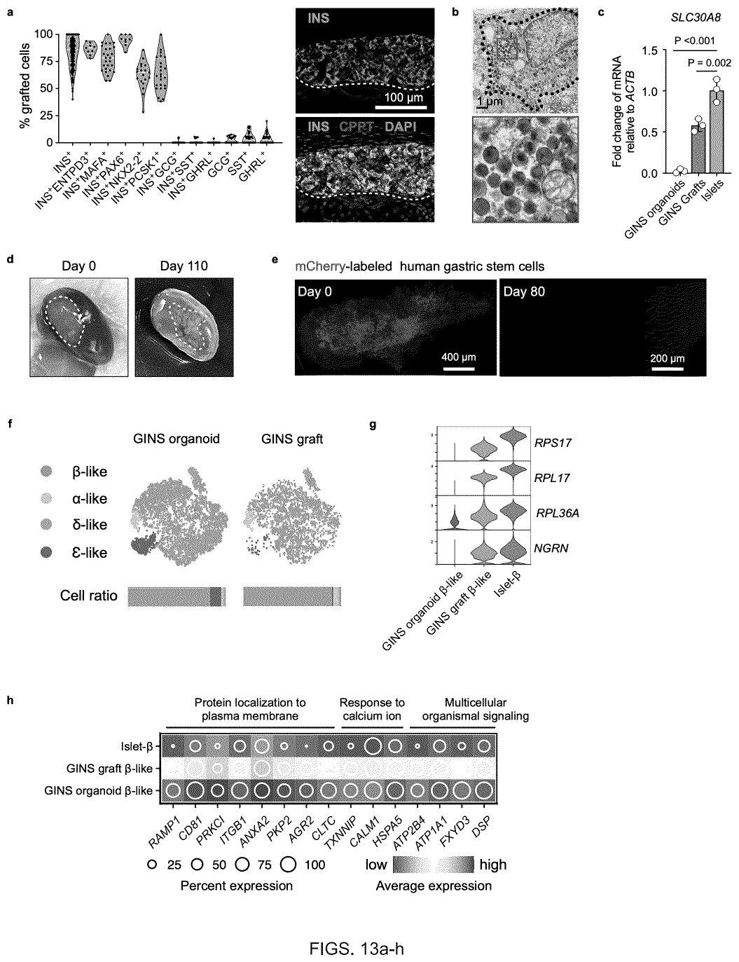

To In order to evaluate the longevity and functionality of GINS cells in vivo, GINS cells were transplanted under the kidney capsule of immune-compromised NSG mice (0.8 million cells per mouse). The grafts were examined at 2, 4, and 6 months. At each time point, the grafts contained abundant INS⁺ cells perfused with CD31⁺ vasculature and minor populations of GCG⁺, SST⁺, and GHRL⁺ cells (FIG. 4a, FIG. 13a). Grafted GINS cells expressed PAX6, NKX2-2, PCSK1, and the adult β-cell marker ENTPD3 (also known as NTPDase3) (Saunders et.al., 2019, Cell Metab 29, 745-754) (FIG. 4a). Electron microscopy showed that the electron-dense granules of the GINS cells were not fully condensed (FIG. 13b), likely reflecting lower levels of SLC30A8 (FIG. 13c), the activity of which was required for the granule morphology (Lemaire et.al., Proc Natl Acad Sci USA 106, 14872-14877). Loss of SLC30A8 was associated with protection against type 2 diabetes (Flannick, et.al., 2014, Nat Genet 46, 357-363; Dwivedi, et.al., Nat Genet 51, 1596-1606).

The majority of the GINS grafts showed glucose-stimulated insulin secretion (FIG. 4b). Accordingly, transplantation of 6-8 million GINS cells from donor #6 into NSG mice rendered diabetic by streptozotocin (STZ) rapidly suppressed hyperglycemia and maintained glucose homeostasis for over 100 days, until removal of the grafts by nephrectomy (FIG. 4c, FIG. 13d). A second cohort of mice transplanted with organoids from a different donor (#10, 6 million cells per mouse) yielded similar results although the glycemic control was less tight (FIG. 4c). Glucose tolerance improved significantly in both engrafted groups (FIG. 4d). Importantly, no proliferating cells were detected within the grafts at any time point (FIG. 4e). To directly evaluate the fate of hGSCs upon transplantation, 0.5 million undifferentiated mCherry-labeled hGSCs were engrafted under the kidney capsule of 6 NSG mice. After 80 days, no surviving cells were detected at the graft sites (FIG. 13e), consistent with the well-documented reliance of hGSCs on high WNT signaling to survive (van der Flier et.al., 2009, Annu Rev Physiol 71, 241-260; Yan et.al., Nature 545, 238-242). It was concluded that GINS cells, derived from human gut stem cells, can be long-lived and functional and pose little risk of uncontrolled proliferation after transplantation.

Comparison of GINS single-cell transcriptomes before and after transplantation (6,242 cells in vitro, 3,502 cells in vivo at 3 months post transplantation) showed molecular changes consistent with maturation, including enhanced expression of NKX2-2, PAX6, UCN3, and ENTPD3 and reduced TFF2 and GHRL (FIG. 3f). Key ribonucleoproteins were up-regulated whereas several pathways elevated in cultured GINS cells were down-regulated after transplantation (FIGS. 13f-h). Insulin expression, while variable in cultured GINS cells, became notably more uniform in the grafted cells (FIG. 4f). Correlation coefficient analysis based on the top 2,000 variable genes showed that GINS transcriptomes became more homogeneous after transplantation, a characteristic shared with islet β cells (FIG. 4g). These results together indicated molecular maturation of GINS cells after transplantation.

### Example 5: Developmental Trajectory of GINS Cells

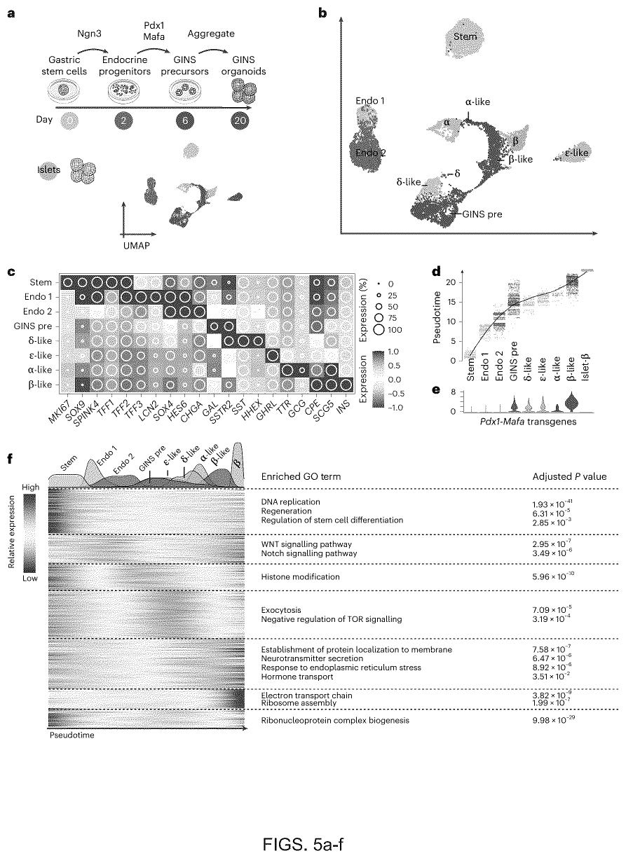

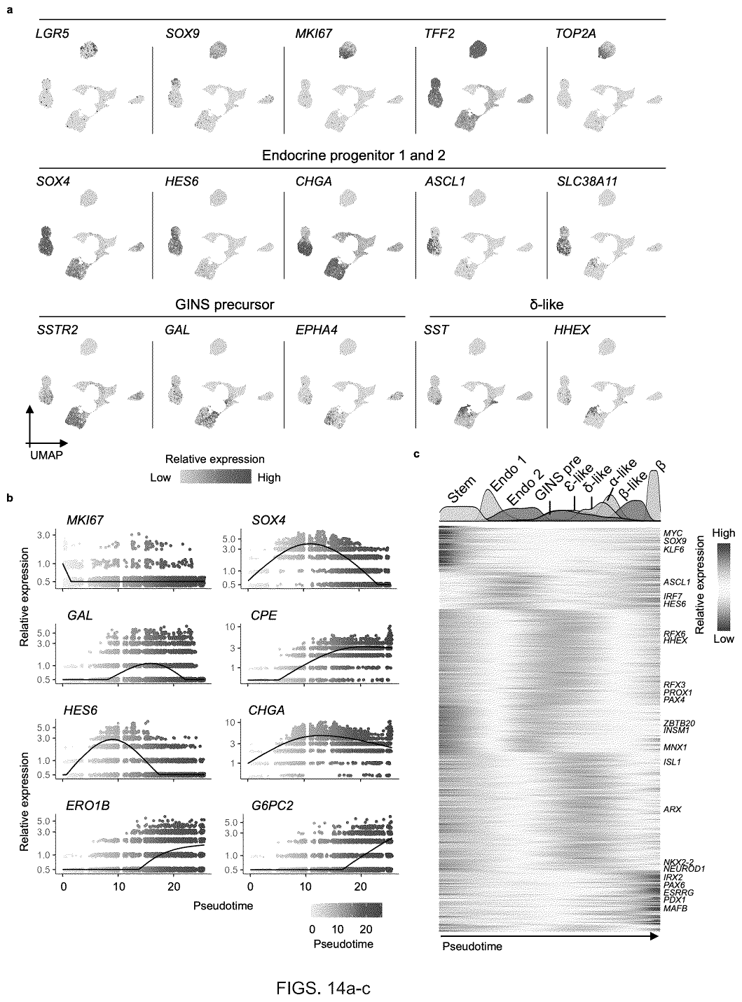

hGSCs normally produce stomach-specific cells including mucus- and acid-secreting cells. To understand how their differentiation path is rerouted in GINS formation, scRNA-seq was used to sample key stages in GINS derivation and reconstructed the developmental trajectory with pseudotime ordering. In total, 9,544 high-quality single cell transcriptomes were collected from four samples: hGSCs, endocrine progenitors (2 days after NGN3 activation), GINS precursors (4 days after PDX1-MAFA expression), and GINS organoids (14 days after aggregation) (FIG. 5a). Clustering analysis showed one stem cell (SOX9^{High}TFF2^{High}LGR5^{High}) two endocrine progenitors (SOX4^{High}CHGA^{Low} and SOX4^{High}CHGA^{High}), one GINS precursor (GAL^{High}SSTR2High), and four endocrine cell populations (FIGS. 5b, c, FIG. 14a) along pseudotemporal progression (FIG. 5d, FIG. 14b). Notably, somatostatin-expressing δ-like cells emerged ahead of the other endocrine cells (FIGS. 5a, 5b, 5d). Pdx1 and Mafa transgene expression was significantly higher in GINS cells than the other endocrine cells, suggesting higher transgenes promoted β-cell fate at the expense of the other endocrine cell types (FIG. 5e). Gene Ontology analysis showed rapid down-regulation of stem cell and proliferative pathways upon endocrine differentiation (FIG. 5f). WNT and NOTCH signaling were active in endocrine precursors whereas histone modification was associated with the GINS precursors. Functional pathways characteristic of β-cells such as hormone transport and secretion, and mitochondria and ribosome activities, emerged last (FIG. 5f).

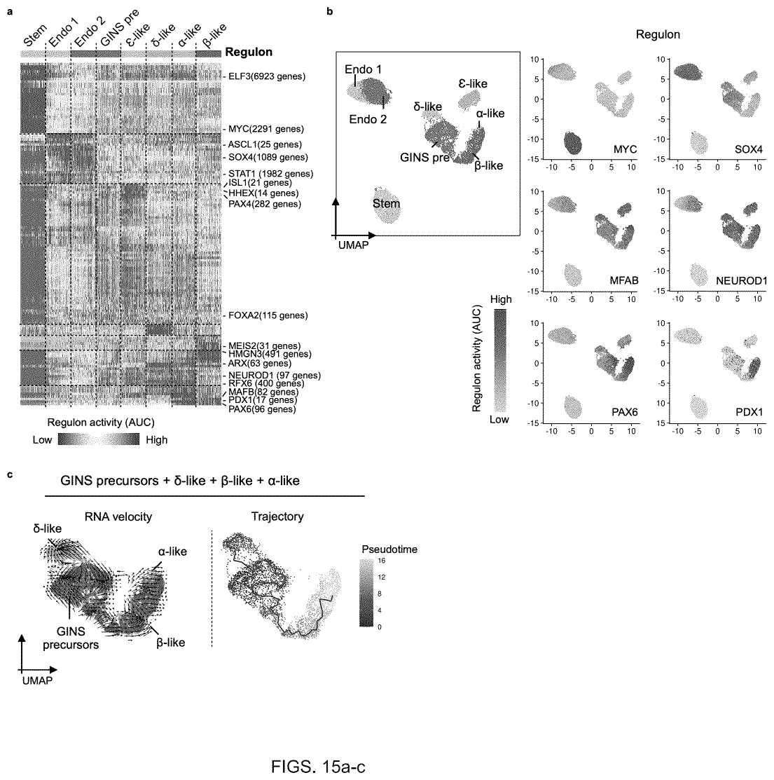

Waves of transcription factor (TF) activations accompanied the progression from hGSCs to GINS cells, likely orchestrating the stepwise acquisition of GINS fate (FIG. 14c). Regulon analysis showed active ASCL1 and SOX4 regulons in endocrine progenitors (FIGS. 15a, 15b). Early-activating GINS regulons included ones for HHEX, PAX4, and ISL1, while late-activating regulons included ones for RFX6, PAX6, PDX1 and MAFB (FIGS. 15a, 15b).

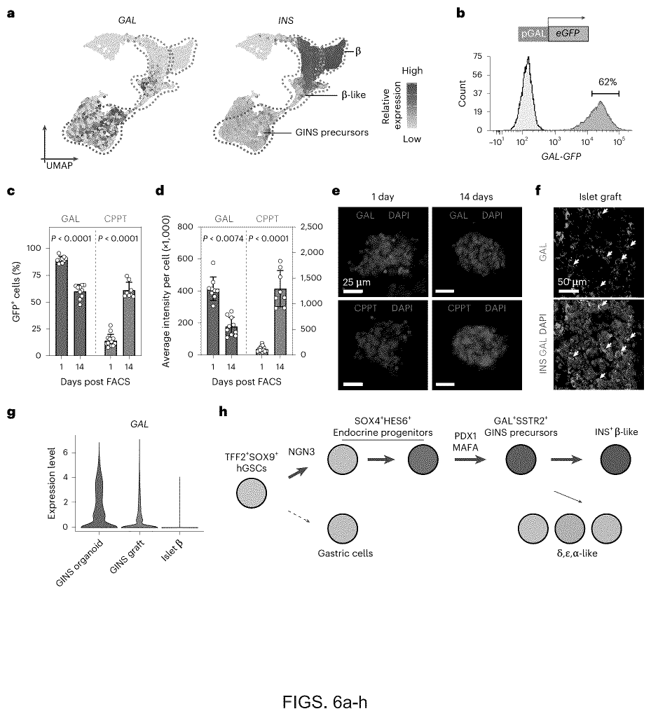

Pseudotime ordering and RNA velocity analysis suggest that β-like cells descended from GINS precursors (FIG. 5d, FIG. 15c), which expressed several markers including SSTR2 and GALANIN (GAL), a neuropeptide predominantly expressed in the nervous system (FIG. 6a). To evaluate whether GAL⁺ precursors can give rise to CPPT⁺ cells, a reporter construct was made in which GFP expression was driven by human GAL promoter and integrated this construct into a hGSC line (FIG. 6b). Six days post differentiation, GFP expression was activated, consistent with the appearance of GINS precursors at this stage. Then purified the GFP^{high} cell fraction was purified by flow cytometry and aggregated the cells into organoids. After overnight culture, the nascent GINS organoids contained predominant GAL⁺ cells (89.9%±3.0%) whereas a small fraction (14.2%±5.9%) had barely detectable levels of CPPT (FIGS. 6c-e). After 14 days of culture, the percentage of GAL⁺ cells and the average GAL staining intensity decreased while the percentage of CPPT⁺ cells rose to 61.2%±7.5% and CPPT staining intensity significantly increased (FIGS. 6c-e). These data showed that GAL⁺ cells can indeed serve as precursors to CPPT⁺ GINS cells. It is noted that GALANIN is expressed in human islets, including some β-cells (FIG. 6f). Upon transplantation and maturation, GAL expression in GINS cells further decreased (FIG. 6g). Altogether, the data supported a model in which sequential Ngn3 and Pdx1-Mafa expression triggered waves of TF activations that led gut cells onto a distinctive differentiation path, including a galanin-expressing precursor, before adopting GINS identity (FIG. 6h).

TABLE 1
Cell type signature gene sets

| Gastric signature | β-cell signature |
|---|---|
| APLP1 | AACS |
| AZGP1 | AARS1 |
| CA9 | ABAT |
| CDKN1C | ABCA5 |
| CHIA | ABCC8 |
| CST3 | ABCG1 |
| CXCL17 | ABHD10 |
| DCXR | ABHD3 |
| EEF2 | ABR |
| ELAPOR1 | ABR |
| ELL2 | ABR |
| ENAH | ABRACL |
| FMOD | AC099548.2 |
| FNDC3B | AC099548.2 |
| FTH1 | ACAT1 |
| FTL | ACLY |
| GLUL | ACVR1 |
| GPER1 | ACVR1B |
| GPRC5C | ACVR1C |
| HMGCS2 | ADCYAP1 |
| IER2 | ADH5 |
| IGFBP2 | ADM |
| IL33 | ADRA2A |
| IRX2 | AFF1 |
| ITIH5 | AFF4 |
| ITLN1 | AGTPBP1 |
| JUN | AHCTF1 |
| LEPR | AKAP11 |
| LINC00261 | AKT3 |
| LIPF | AKT3 |
| MECOM | AKTIP |
| METTL7A | ALCAM |
| MT1F | ALDOC |
| MT1G | ALG2 |
| MT1X | ALKBH5 |
| MT2A | AMPD2 |
| NEDD4L | ANKH |
| NR2F2 | ANKMY2 |
| P4HB | ANKRD12 |
| PABPC4 | ANKRD65 |
| PDIA2 | ANO5 |
| PGA5 | ANP32B |
| PGC | ANP32E |
| PLIN5 | AP1AR |
| PRDX4 | AP3B1 |
| RPLP1 | APC |
| SEC62 | APCDD1L-DT |
| SLC12A2 | APPL1 |
| SOX4 | APPL2 |
| SSR4 | ARFGEF2 |
| TMED6 | ARG2 |
| TMEM97 | ARHGAP35 |
| TSC22D1 | ARHGAP5 |
| UGT2B15 | ARHGEF3 |
| XBP1 | ARHGEF9 |
| XYLT2 | ARL4D |
| ABCC3 | ARL6IP5 |
| ABCC5 | ARL8B |
| ABHD2 | ARMCX3 |
| ABLIM1 | ARPP19 |
| ACTB | ASAP1 |
| ACTG1 | ASH1L |
| ACTN4 | ATF6 |
| AGR2 | ATG3 |
| AHNAK | ATP1B2 |
| AKR1B10 | ATP2A3 |
| AKR1C1 | ATP6V0A1 |
| AKR1C2 | ATP6V0E2 |
| AKR1C3 | ATP6V1A |
| ALDH2 | ATP6V1B2 |
| ALDH3A1 | ATP6V1C1 |
| ALDH3A2 | ATP6V1D |
| ANG | ATP6V1E1 |
| ANXA10 | ATP6V1G1 |
| ARPC1A | ATP8A1 |
| BCAS1 | ATRNL1 |
| BLVRB | ATRX |
| CA2 | ATXN2 |
| CALM2 | AZI2 |
| CAPG | BAG3 |
| CAPN5 | BBS2 |
| CAPN8 | BBX |
| CAPN9 | BCL2L2 |
| CAST | BCL9 |
| CBR1 | BEX1 |
| CCND1 | BEX2 |
| CD2AP | BEX4 |
| CD63 | BHLHE41 |
| CEACAM5 | BLOC1S2 |
| CES2 | BMP5 |
| CFL1 | BNIP3 |
| CLDN18 | BNIP3L |
| CLIC1 | BRD7 |
| CLTB | BRI3 |
| CMBL | BTBD1 |
| COX8A | BTBD3 |
| CRIP1 | BTF3L4 |
| CSTB | BTG3 |
| CTNNA1 | BTG3 |
| CTSE | C11orf1 |
| CYP2C18 | C12orf76 |
| CYP2S1 | C14orf132 |
| CYP3A5 | C15orf61 |
| CYSTM1 | C16orf72 |
| DDX60 | C1D |
| DSC2 | C1orf43 |
| DYNLL1 | C21orf91 |
| EFHD2 | C2CD5 |
| EFNB2 | C2orf69 |
| ETNK1 | C4orf3 |
| FA2H | CABP7 |
| FAM120A | CACNA1D |
| FBLIM1 | CACNA1H |
| FCGBP | CACNA2D1 |
| FHL2 | CACYBP |
| FLNB | CADM1 |
| FOXA2 | CADPS |
| FOXQ1 | CAMK2N1 |
| FSIP2 | CAMLG |
| FXYD3 | CANX |
| GALE | CANX |
| GALNT6 | CAPN13 |
| GKN1 | CAPN7 |
| GKN2 | CASC3 |
| GPX2 | CASR |
| GSN | CBX4 |
| GSTP1 | CBX7 |
| HPGD | CCNI |
| HSPB1 | CCSER2 |
| ID1 | CCT4 |
| IFI27 | CD200 |
| IL1R2 | CDC37L1 |
| IL1RN | CDC42BPA |
| ITGB4 | CDC42EP3 |
| JPT1 | CDC5L |
| JUP | CDCP1 |
| KDELR2 | CDIP1 |
| KLF3 | CDIP1 |
| KLF4 | CDK5R1 |
| KRT18 | CDK6 |
| KRT19 | CDKN1A |
| KRT8 | CDKN1C |
| LGALS3 | CDKN1C |
| LGALS4 | CDV3 |
| LGALS9C | CEBPG |
| LINC01133 | CELF4 |
| LMNA | CENPC |
| LMO7 | CERK |
| MAL2 | CFAP36 |
| MANF | CFL2 |
| MAPK3 | CHD7 |
| MGLL | CHD9 |
| MGST3 | CHL1 |
| MUC1 | CHN1 |
| MUCL3 | CIRBP |
| NET1 | CISD2 |
| NQO1 | CLASP2 |
| PERP | CLCN3 |
| PFN1 | CLCN4 |
| PHGR1 | CLGN |
| PLA2G10 | CLIP1 |
| PLAC8 | CLIP4 |
| PLXNB2 | CLTA |
| PRDX1 | CLTC |
| PSCA | CMTM8 |
| PTGR1 | CNBP |
| RAB27A | CNP |
| RAB27B | CNRIP1 |
| RAC1 | COBLL1 |
| RALA | COMMD9 |
| RASSF6 | COPRS |
| RBM47 | COPS2 |
| REG4 | COPS7A |
| REP15 | COX7A2L |
| RHOA | CPD |
| RNASE1 | CPEB4 |
| S100A11 | CRACD |
| S100A14 | CREB3L2 |
| S100A16 | CREBL2 |
| S100A6 | CREBRF |
| S100P | CRMP1 |
| SFN | CSDE1 |
| SH3BGRL2 | CYFIP2 |
| SH3BGRL3 | CYP2U1 |
| SLC44A1 | CYYR1 |
| SLC44A2 | DARS1 |
| SLC5A5 | DBI |
| SLC7A8 | DDIT3 |
| SLC9A1 | DDR1 |
| SMPD3 | DDR1 |
| SOSTDC1 | DDR1 |
| SPINK1 | DDR1 |
| SPINT2 | DDR1 |
| SPTSSB | DDR1 |
| SULT1C2 | DDR1 |
| SYTL2 | DDX24 |
| TACSTD2 | DDX24 |
| TAGLN2 | DDX3Y |
| TCEAL9 | DENND5B |
| TCF7L2 | DGKD |
| TENT5A | DGKD |
| TESC | DGKE |
| TFF1 | DHRS2 |
| TFF2 | DHRS7 |
| TMEM54 | DICER1 |
| TMSB4X | DIP2C |
| TPM1 | DLGAP4 |
| TPM4 | DLK1 |
| TRNP1 | DMXL2 |
| TSPAN1 | DNAH5 |
| TSPAN3 | DNAJB1 |
| TSPAN8 | DNAJB9 |
| TST | DNAJC12 |
| TXN | DNAJC3 |
| UGDH | DNAJC6 |
| VAMP8 | DOCK10 |
| VILL | DPP6 |
| VSIG1 | DPYSL2 |
| VSIG2 | DSP |
| YWHAZ | DST |
| ZBTB7C | DSTYK |
| AC008397.1 | DTX3 |
| ACIN1 | DYNC1H1 |
| ACTR2 | DYNLT3 |
| ADAM15 | ECPAS |
| ADD3 | EDARADD |
| ADH1C | EDN3 |
| AGR3 | EEF2 |
| AKAP1 | EFNA5 |
| ANO1 | EFR3A |
| ANXA2 | EIF1B |
| AP1M2 | EIF3E |
| AP2B1 | EIF3H |
| ARF1 | EIF4A2 |
| ARF5 | EIF4B |
| ARHGAP21 | EIF4G3 |
| ARHGDIA | ELAVL4 |
| ARL6IP1 | ELMO1 |
| ARPC2 | ELMO2 |
| ASAP2 | ELP4 |
| ASPM | EML6 |
| ATP1A1 | ENDOD1 |
| ATP2A3 | ENO1 |
| ATP5IF1 | ENO2 |
| ATP5ME | ENPP5 |
| ATP8B1 | ENTPD3 |
| AUP1 | EPB41L1 |
| B4GALT1 | EPB41L3 |
| BACE2 | EPB41L4A-AS1 |
| BAZA | EPM2AIP1 |
| BCAP31 | EPRS1 |
| BHLHE40 | ERICH5 |
| CALM3 | ERO1B |
| CALR | ERRFI1 |
| CANX | ESD |
| CAP1 | ETNK1 |
| CAPNS1 | EXOC2 |
| CAPZB | EXOC5 |
| CCDC25 | EXPH5 |
| CCNB1 | FAM107A |
| CCT5 | FAM135A |
| CD151 | FAM174B |
| CDC37 | FAM200B |
| CDC42EP5 | FAM219A |
| CDH1 | FAM222A |
| CDS1 | FAM8A1 |
| CENPE | FASN |
| CENPF | FBXL16 |
| CENPV | FBXL17 |
| CHCHD2 | FBXO17 |
| CKAP4 | FBXO17 |
| CKS2 | FBXO33 |
| CLMN | FBXO7 |
| CORO2A | FFAR4 |
| COX6B1 | FGF9 |
| CREB3L1 | FKBP4 |
| CRELD2 | FKBP5 |
| CTTN | FLOT1 |
| CYC1 | FLOT1 |
| CYP1B1 | FLOT1 |
| CYP4F12 | FLOT1 |
| CYP51A1 | FLOT1 |
| DBI | FLOT1 |
| DDX18 | FLOT1 |
| DDX21 | FLOT1 |
| DHCR24 | FMN2 |
| DHFR | FNIP1 |
| DNAJB11 | FNIP2 |
| DNM2 | FOXA2 |
| DSP | FOXJ3 |
| DST | FOXN3 |
| EBP | FOXO1 |
| EDEM3 | FRA10AC1 |
| EI24 | FTH1 |
| EIF2S2 | FTH1P3 |
| EIF3A | FTL |
| EIF3J | FXR1 |
| EIF4E2 | FYN |
| EIF4G1 | FYTTD1 |
| EIF5B | G3BP1 |
| EIF6 | G3BP2 |
| ELF3 | G6PC2 |
| ELOVL6 | G6PC2 |
| ENO1 | GABARAP |
| ENSA | GABARAPL2 |
| EPCAM | GAD2 |
| EPS8L3 | GAS5 |
| ESRP1 | GCC2 |
| ETFB | GDA |
| EZR | GDAP1 |
| FABP5 | GEM |
| FAM177B | GFPT1 |
| FASN | GHITM |
| FBP1 | GIGYF2 |
| FER1L6 | GLCCI1 |
| FERMT1 | GLIS2 |
| FKBP2 | GLIS2 |
| FMN1 | GLIS3 |
| GADD45A | GLT8D2 |
| GAPDH | GNA13 |
| GARS1 | GNAO1 |
| GFUS | GNAS |
| GIPC1 | GNG4 |
| GMDS | GNG4 |
| GNA11 | GNG7 |
| GNB2 | GNPDA1 |
| GNL3 | GOLGA4 |
| GOLGA4 | GOLGA8A |
| GSTO1 | GOLGB1 |
| H2AJ | GPM6A |
| H2AZ1 | GPR158 |
| H4C3 | GPRASP1 |
| HDGF | GPSM1 |
| HDLBP | GREM1 |
| HES1 | GREM1 |
| HMGB1 | GREM1 |
| HMGB2 | GREM2 |
| HMGCS1 | GRIA4 |
| HNRNPA2B1 | GRPEL2 |
| HNRNPAB | GSE1 |
| HNRNPF | GSN |
| HNRNPM | GTF3C1 |
| HSP90AA1 | GYG1 |
| HSP90AB1 | HABP4 |
| HSP90B1 | HACD3 |
| HSPA5 | HADH |
| HSPD1 | HAX1 |
| HSPH1 | HCFC2 |
| HYOU1 | HDAC9 |
| IBTK | HECTD1 |
| IER3 | HECTD4 |
| INF2 | HERPUD1 |
| ITGA6 | HIVEP2 |
| ITGB1 | HLF |
| ITPR3 | HLTF |
| KCNE3 | HMGCLL1 |
| KDELR1 | HNRNPC |
| KDELR3 | HOOK1 |
| KHSRP | HOPX |
| KIAA1522 | HPS4 |
| KIF1C | HS6ST1 |
| KIF5B | HSP90AA1 |
| KLF2 | HSP90AB1 |
| KLF5 | HSPA13 |
| LAD1 | HSPA1A |
| LAMA3 | HSPA1A |
| LAMB3 | HSPA1A |
| LARP1 | HSPA1A |
| LARP1B | HSPA1A |
| LASP1 | HSPA1B |
| LDLR | HSPA1B |
| LFNG | HSPA1B |
| LIMA1 | HSPA1B |
| LLGL2 | HSPA1B |
| LMAN2 | HSPA4L |
| LMO4 | HSPA8 |
| LPCAT4 | HSPA9 |
| LRRFIP1 | HSPD1 |
| MACROH2A1 | HSPH1 |
| MARCKS | HUWE1 |
| MCU | IAPP |
| ME1 | ID1 |
| MESD | ID4 |
| MGST2 | IER3 |
| MIA3 | IER3 |
| MICU1 | IER3 |
| MIDN | IER3 |
| MISP | IER3 |
| MKI67 | IER3 |
| MLEC | IGBP1 |
| MLPH | IGF1R |
| MRPL14 | IGSF1 |
| MRPS35 | IGSF11 |
| MTDH | IL17RB |
| MVD | IL6R |
| MVP | ILF3-DT |
| MYDGF | INA |
| MYH14 | ING2 |
| MYL6 | INPP5F |
| MYO10 | INS |
| MYO1A | INSIG1 |
| MYO5C | INSM1 |
| MYOF | IPO7 |
| NANS | IQSEC1 |
| NCL | IRF2BP2 |
| NCOR1 | IRS2 |
| NDUFS6 | ISCU |
| NFE2L2 | ISOC1 |
| NFIC | ITPR3 |
| NIBAN2 | JAZF1 |
| NKX6-2 | JKAMP |
| NMU | JUND |
| NOP56 | KAT2B |
| NR2F6 | KAT6B |
| NUCKS1 | KAT6B |
| NUSAP1 | KATNAL1 |
| OSBPL7 | KCMF1 |
| PABPC1 | KCNG3 |
| PALLD | KCNH2 |
| PAQR8 | KCNJ11 |
| PDIA4 | KCNK16 |
| PDIA6 | KCNK17 |
| PDLIM1 | KCNK3 |
| PEBP1 | KCNMA1 |
| PHACTR2 | KCTD13 |
| PHLDA2 | KDM3A |
| PKP3 | KDM7A |
| PLEC | KIAA2026 |
| PLEK2 | KIDINS220 |
| PLEKHA6 | KIF13A |
| PLLP | KIF1A |
| PLP2 | KIF21A |
| PLXNA2 | KIF3A |
| POMP | KIF3B |
| PPARG | KIF5B |
| PPDPF | KIF5C |
| PPFIBP1 | KIF5C |
| PPIA | KLF10 |
| PPM1G | KLF13 |
| PRC1 | KLF13 |
| PRKCD | KLHDC10 |
| PRPF38B | KLHDC8A |
| PSD3 | KLHL12 |
| PSMA4 | KLHL2 |
| PSMA7 | KLHL42 |
| PSMD11 | KMT2C |
| PSMD7 | KTN1 |
| PTBP1 | LARP1 |
| PTGES3 | LDOC1 |
| PTMA | LETMD1 |
| PTPRF | LINC01128 |
| PTTG1 | LMAN1 |
| PTTG1IP | LMO2 |
| PYCR1 | LNPK |
| RAB11FIP1 | LONRF2 |
| RAB2A | LPIN1 |
| RAB3D | LRPPRC |
| RALBP1 | LRRTM3 |
| RAN | LYSMD2 |
| RASEF | LYST |
| RBM25 | MADD |
| RBM3 | MAFA |
| RETSAT | MAFB |
| RNU4ATAC | MAFG |
| RPL35 | MAGI2-AS3 |
| RPL36AL | MAN1A2 |
| RPN1 | MAN1C1 |
| RPN2 | MAN2A2 |
| RPS24 | MAP1A |
| RPS8 | MAP1B |
| RRBP1 | MAP1LC3A |
| S100A10 | MAP2 |
| SCD | MAP3K2 |
| SDC1 | MAP6 |
| SDC4 | MAPRE1 |
| SDF2L1 | MAPRE2 |
| SEC61A1 | MAPT |
| SEC61G | MAPT |
| SEC63 | MAPT |
| SEM1 | MARK1 |
| SEMA3B | MAST4 |
| SERBP1 | MATR3 |
| SERF2 | MATR3 |
| SERPINB1 | MBD1 |
| SERPINB5 | MBP |
| SF3B5 | MCC |
| SGSM3 | MCUB |
| SH3RF1 | MDM2 |
| SLC16A1 | ME1 |
| SLC39A11 | MEG3 |
| SLK | MEGF9 |
| SLTM | MEIS2 |
| SMC4 | METTL5 |
| SMIM14 | MGAT4A |
| SNRPB | MIA3 |
| SNRPD1 | MIR4458HG |
| SNRPF | MIR7-3HG |
| SNRPG | MIS18BP1 |
| SPINK5 | MKRN2 |
| SPTBN1 | MLXIPL |
| SRRM1 | MNX1 |
| SRSF1 | MPHOSPH8 |
| SRSF3 | MPP1 |
| STMN1 | MPP6 |
| SYK | MRPL34 |
| SYNGR2 | MRPL49 |
| TACC3 | MRPS18B |
| TALDO1 | MRPS18B |
| TCERG1 | MRPS18B |
| TES | MRPS18B |
| TFCP2L1 | MRPS18B |
| TIMP1 | MRPS18B |
| TK1 | MRPS18B |
| TKT | MSANTD4 |
| TLCD2 | MT1F |
| TLN2 | MT1X |
| TM9SF3 | MT2A |
| TMED2 | MTERF2 |
| TMED9 | MTERF4 |
| TMPRSS4 | MTMR6 |
| TMSB10 | MTRNR2L1 |
| TOP1 | MTRNR2L2 |
| TOP2A | MTRNR2L8 |
| TPM3 | MTSS1 |
| TPX2 | MTSS2 |
| TRIM2 | MTURN |
| TSPAN15 | MTUS2 |
| TSPO | MXI1 |
| TTC39A | MXRA7 |
| TUBA1B | MYCBP2 |
| TUBA1C | MYH14 |
| TUBB | MYO1D |
| TUBB4B | MYO3A |
| TXNRD1 | MYO5A |
| U2AF1 | NANOS1 |
| UBE2C | NAP1L1 |
| USH1C | NAP1L2 |
| VASP | NAP1L5 |
| VCP | NAPA |
| VMP1 | NAV2 |
| VPS35 | NBEA |
| WASF2 | NCKAP1 |
| YBX1 | NCOA3 |
| YWHAB | NCOA4 |
| YWHAH | NCOR2 |
| ZBTB7A | NDFIP1 |
| ALDOC | NDRG1 |
| ARHGEF2 | NEBL |
| ARL14 | NECTIN3 |
| BASP1 | NEFM |
| C15orf48 | NEO1 |
| CD164 | NEU1 |
| CD59 | NEU1 |
| COL17A1 | NEU1 |
| CSTA | NEU1 |
| CTNNBL1 | NEU1 |
| DHRS7 | NEU1 |
| DSG2 | NEU1 |
| DUOX2 | NEU1 |
| EHBP1L1 | NEUROD1 |
| EPN1 | NEXMIF |
| EPS8L1 | NFASC |
| FAM102A | NFE2L1 |
| FCGRT | NFIC |
| FOXA3 | NGRN |
| H1-0 | NIN |
| HACD3 | NISCH |
| HIGD1A | NKX2-2 |
| IFI6 | NMNAT2 |
| IGFBP4 | NMRK1 |
| KCNK1 | NORAD |
| KRT20 | NPR2 |
| LINC01559 | NPTX2 |
| LRP10 | NR0B1 |
| MAL | NR1D2 |
| MX2 | NR3C1 |
| MXD1 | NRCAM |
| MYL12A | NRDC |
| MYL12B | NSG1 |
| NAPEPLD | NUCKS1 |
| NEAT1 | NUS1 |
| OAS1 | OCIAD1 |
| OCLN | OLA1 |
| PCSK7 | OLFM1 |
| PGRMC2 | OLMALINC |
| PKIB | ORC3 |
| PLAAT2 | OSBPL8 |
| PNPLA2 | OSTM1 |
| POLD4 | OTULINL |
| PPIF | P2RY1 |
| PRR15 | PAFAH1B1 |
| PSAPL1 | PAIP2 |
| RDH13 | PAN2 |
| RHOC | PAPSS2 |
| RIOK3 | PARVB |
| RIPK4 | PATL1 |
| RNF128 | PCDH7 |
| SCNN1A | PCGF5 |
| SECTM1 | PCLO |
| SLPI | PCM1 |
| SMIM22 | PCMT1 |
| SNX9 | PCSK1 |
| SPATS2L | PDE4DIP |
| SPINT1 | PDE8B |
| SPIRE2 | PDE8B |
| STK24 | PDX1 |
| SYNJ2 | PDZD2 |
| TNFRSF21 | PDZD8 |
| TSKU | PDZRN3 |
| UNC5B | PEBP1 |
| UNC5B-AS1 | PEG3 |
| VEGFA | PELI1 |
| A4GNT | PEPD |
| AQP5 | PER3 |
| C16orf89 | PERP |
| C6orf58 | PFKFB2 |
| CD24 | PFN2 |
| CLU | PGK1 |
| COL11A2 | PGRMC2 |
| DYNC2H1 | PHACTR2 |
| EGR1 | PHC2 |
| ERGIC1 | PHF1 |
| ERN2 | PHF1 |
| ETV5 | PHF1 |
| FUT9 | PI4KA |
| GOLM1 | PIGA |
| GP2 | PIK3C2A |
| KCNN4 | PIKFYVE |
| LYZ | PIM3 |
| MMP1 | PIP4P2 |
| MSMB | PIPOX |
| MUC6 | PIR |
| NOP53 | PITHD1 |
| NR4A1 | PJA2 |
| PIGR | PKIB |
| PIK3C2G | PKP2 |
| PPP1R1B | PLAGL1 |
| RGMB | PLCB1 |
| SLC4A4 | PLCB4 |
| ACADM | PLCL2 |
| ACADVL | PLCL2 |
| ACAT1 | PLCXD3 |
| AHCYL2 | PLEKHA6 |
| AIFM1 | PLOD2 |
| AK3 | PNMA1 |
| AKR7A3 | PNMA2 |
| AKR7L | PNMA8A |
| ALAD | PPID |
| ALDH1A1 | PPL |
| ALDOB | PPM1A |
| AQP4 | PPM1E |
| ARRDC4 | PPP1R1A |
| ATP1B1 | PPP1R2 |
| ATP4A | PPP2CB |
| ATP4B | PPT1 |
| ATP5F1A | PRDM4 |
| ATP5F1B | PREPL |
| ATP5F1C | PRKACB |
| ATP5F1D | PRKAG2 |
| ATP5MC1 | PRKAR2B |
| ATP5MC3 | PRKAR2B |
| ATP5MD | PRKCH |
| ATP5MF | PRMT2 |
| ATP5MG | PRNP |
| ATP5MPL | PRPF8 |
| ATP5PB | PRPF8 |
| ATP5PD | PRPS1 |
| ATP5PF | PRSS23 |
| ATP5PO | PRUNE2 |
| ATP6V1H | PRXL2A |
| AUH | PSAP |
| BCKDHB | PSD3 |
| BOLA3 | PSIP1 |
| Clorf116 | PSMB1 |
| CALM1 | PSMB1 |
| CBLIF | PSMC6 |
| CCKBR | PSMD1 |
| CHCHD10 | PTAR1 |
| CHP1 | PTBP3 |
| CHPT1 | PTCH1 |
| CKB | PTEN |
| CKMT2 | PTEN |
| CLIC6 | PTGES3 |
| CMTM4 | PTPN1 |
| CNTD1 | PTPN11 |
| COA3 | PTPN3 |
| COX17 | PTPRJ |
| COX4I1 | PTPRN |
| COX5A | PURA |
| COX5B | PURB |
| COX6A1 | RAB11FIP1 |
| COX6C | RAB11FIP5 |
| COX7A2 | RAB12 |
| COX7A2L | RAB15 |
| COX7B | RAB22A |
| COX7C | RAB29 |
| CPA2 | RAB39B |
| CPEB4 | RAB3B |
| CS | RAB3GAP1 |
| CYCS | RAB3IP |
| CYFIP2 | RAB5A |
| DBT | RABEP1 |
| DECR1 | RABGGTB |
| DLD | RAD17 |
| DNER | RAD17 |
| DRD5 | RAD50 |
| DUSP19 | RALGPS1 |
| DUSP4 | RANBP2 |
| ECHS1 | RAP1GAP2 |
| ECI2 | RAPH1 |
| EIF5A2 | RASA4 |
| ESRRG | RASD1 |
| ETFRF1 | RASGRF1 |
| ETNPPL | RASGRP1 |
| FAM107B | RASSF6 |
| FAM162A | RBBP8 |
| FGA | RBP4 |
| FGB | RCAN2 |
| FGD4 | RCAN3 |
| FGG | RCBTB2 |
| FH | RCOR3 |
| FILIP1L | REPIN1 |
| FNDC5 | RERE |
| GOS2 | RETREG2 |
| GABARAPL1 | RGL1 |
| GBGT1 | RGS16 |
| GHITM | RHEB |
| GLOD4 | RHOQ |
| GMPR | RIMKLA |
| GOT1 | RMND5A |
| GOT2 | RNF13 |
| GPC3 | RNF130 |
| GPR155 | RNF157 |
| GPT2 | RNF187 |
| GRIA4 | RNF187 |
| GSTA1 | RNF19B |
| GUCA2B | RNF2 |
| HADHB | RNF6 |
| HOPX | RNMT |
| HPCAL1 | ROBO1 |
| HSPA9 | ROBO2 |
| IDH2 | ROCK1 |
| IGFBP5 | ROR1 |
| INSIG1 | RORA |
| ISCU | RPA2 |
| KCNE2 | RPH3AL |
| KCNJ16 | RPH3AL |
| KCNQ1 | RPH3AL |
| KIAA1191 | RPL17 |
| LDHB | RPL3 |
| LIFR | RPL31 |
| LINC00671 | RPL36AL |
| LRIG1 | RPL37 |
| MDH1 | RPL4 |
| MDH2 | RPL5 |
| ME3 | RPL7A |
| MFSD4A | RPL7A |
| MGAT4B | RPS23 |
| MICALL1 | RPS3 |
| MICOS10 | RPS4X |
| MPC1 | RRAGA |
| MPC2 | RRAGC |
| MPP7 | RRAGD |
| MRPL34 | RRM2B |
| MRPL41 | RRN3 |
| MTURN | RRN3 |
| MYO19 | RTCB |
| NCKAP1 | RTN4 |
| NDUFA1 | RUFY3 |
| NDUFA10 | RUNX1T1 |
| NDUFA4 | SAMD3 |
| NDUFA5 | SARS1 |
| NDUFA6 | SBDS |
| NDUFA7 | SBNO1 |
| NDUFA8 | SC5D |
| NDUFA9 | SCAPER |
| NDUFAB1 | SCD |
| NDUFB10 | SCD5 |
| NDUFB2 | SCG3 |
| NDUFB3 | SCGN |
| NDUFB4 | SCN1B |
| NDUFB5 | SEC11C |
| NDUFB7 | SEC63 |
| NDUFB8 | SELENOW |
| NDUFB9 | SEMA5A |
| NDUFC1 | SEMA6D |
| NDUFS1 | SERINC1 |
| NDUFS2 | SERPINB4 |
| NDUFS3 | SESN1 |
| NDUFS4 | SETBP1 |
| NDUFS8 | SETD3 |
| NDUFV1 | SETD7 |
| NDUFV2 | SGMS2 |
| NECAB3 | SH3GL2 |
| NNT | SHISAL1 |
| ODAM | SHISAL2B |
| OXCT1 | SHOC2 |
| PAFAH2 | SHTN1 |
| PBXIP1 | SIPA1L2 |
| PDGFD | SIX2 |
| PDHA1 | SIX3 |
| PDHB | SLAIN2 |
| PDHX | SLC11A2 |
| PHLDA1 | SLC16A9 |
| PINK1 | SLC18A2 |
| PKM | SLC1A4 |
| PLEKHB2 | SLC25A34 |
| PP7080 | SLC25A36 |
| PPP2R3A | SLC25A4 |
| PPP2R5A | SLC29A1 |
| PRDM16-DT | SLC2A1 |
| PRDX2 | SLC2A13 |
| PRDX3 | SLC2A2 |
| PRDX5 | SLC30A8 |
| PRKACB | SLC39A14 |
| PRKAR1A | SLC4A10 |
| PSAP | SLC4A7 |
| PTGER3 | SLC6A17 |
| PXMP2 | SLC6A6 |
| RAB11FIP2 | SLC7A1 |
| RAB4A | SLK |
| RAP1GAP | SMAD7 |
| RAP1GAP2 | SMAD9 |
| RBPMS2 | SMIM29 |
| REXO2 | SNAP91 |
| SDHB | SNHG32 |
| SDHC | SNHG32 |
| SGK1 | SNHG32 |
| SIGLEC11 | SNHG32 |
| SIK2 | SNHG32 |
| SLC16A7 | SNHG7 |
| SLC25A3 | SNX29 |
| SLC25A4 | SNX9 |
| SLC25A5 | SOBP |
| SLC2A12 | SOCS2 |
| SLC4A2 | SOCS7 |
| SLC9A3R1 | SOCS7 |
| SMIM11A | SORL1 |
| SOD1 | SPECC1L |
| STX12 | SPEG |
| SUCLG1 | SPIN1 |
| SUCLG2 | SPIRE1 |
| SULT2A1 | SPOPL |
| SYNJ2BP | SPRYD3 |
| TBCA | SQSTM1 |
| TCN1 | SQSTM1 |
| TM7SF2 | SRD5A1 |
| TMBIM6 | SREBF1 |
| TMEM141 | SRPRA |
| TMEM171 | SRXN1 |
| TMEM70 | SSTR5-AS1 |
| TPD52L1 | ST13 |
| TPM2 | ST18 |
| TRIM50 | ST3GAL5 |
| TXNDC17 | ST8SIA3 |
| UBL3 | STAG2 |
| UQCR10 | STMN2 |
| UQCR11 | STMN3 |
| UQCRB | STX1A |
| UQCRC1 | STX3 |
| UQCRC2 | STXBP1 |
| UQCRFS1 | STXBP5 |
| UQCRQ | SUCO |
| VDAC1 | SUMO3 |
| VDAC2 | SURF2 |
| VEGFB | SURF2 |
| WBP2 | SURF4 |
| WIPF3 | SURF4 |
| | SUSD4 |
| | SUSD6 |
| | SV2A |
| | SVIP |
| | SYBU |
| | SYNE2 |
| | SYNM |
| | SYT11 |
| | SYT13 |
| | SYT4 |
| | SYT7 |
| | TAB2 |
| | TAF9 |
| | TAF9 |
| | TAGLN3 |
| | TANC2 |
| | TAOK1 |
| | TBC1D24 |
| | TBC1D4 |
| | TBC1D9B |
| | TBC1D9B |
| | TBCC |
| | TBL1XR1 |
| | TCAF1 |
| | TCAF2 |
| | TCEA1 |
| | TERF2 |
| | TERF2IP |
| | TFCP2L1 |
| | TGFBR3 |
| | TGOLN2 |
| | THAP5 |
| | THAP6 |
| | THBD |
| | TIAM1 |
| | TIMP2 |
| | TLE5 |
| | TMCC3 |
| | TMED6 |
| | TMEM109 |
| | TMEM126B |
| | TMEM132B |
| | TMEM150C |
| | TMEM167A |
| | TMEM181 |
| | TMEM33 |
| | TMEM37 |
| | TMEM38B |
| | TMEM64 |
| | TMEM65 |
| | TMOD1 |
| | TMOD2 |
| | TNFRSF11A |
| | TNKS2 |
| | TNS1 |
| | TOGARAM1 |
| | TOMM20 |
| | TOMM34 |
| | TOMM70 |
| | TOR1AIP1 |
| | TPD52 |
| | TPM3 |
| | TPP1 |
| | TPRG1L |
| | TRAPPC8 |
| | TRIB3 |
| | TRIM2 |
| | TRIM23 |
| | TRIM37 |
| | TRIM9 |
| | TRIP11 |
| | TRIQK |
| | TRMT112 |
| | TSHZ1 |
| | TSHZ3 |
| | TSPAN1 |
| | TSPAN13 |
| | TSPAN7 |
| | TSPYL1 |
| | TSPYL2 |
| | TSPYL5 |
| | TTC3 |
| | TTC37 |
| | TTC3P1 |
| | TTC7B |
| | TTLL7 |
| | TUBA4A |
| | TUBB2B |
| | TUNAR |
| | TXNL1 |
| | TXNRD1 |
| | U2SURP |
| | UBC |
| | UBE2QL1 |
| | UBE3C |
| | UBL3 |
| | UBN1 |
| | UBQLN1 |
| | UBQLN2 |
| | UBXN1 |
| | UBXN4 |
| | UCHL1 |
| | UCHL5 |
| | UHMK1 |
| | UNC79 |
| | UNC79 |
| | UNC80 |
| | USF3 |
| | USP1 |
| | USP12 |
| | USP34 |
| | USP48 |
| | USP51 |
| | USP9X |
| | UTRN |
| | VAMP2 |
| | VAPB |
| | VAT1L |
| | VEGFA |
| | VEGFB |
| | VLDLR |
| | VOPP1 |
| | VPS13C |
| | VPS35 |
| | VPS37A |
| | VWDE |
| | WAC |
| | WARS1 |
| | WDR17 |
| | WDR33 |
| | WDR7 |
| | WFS1 |
| | WHAMM |
| | WLS |
| | WNT4 |
| | WSB2 |
| | WSCD2 |
| | XPC |
| | XPOT |
| | YME1L1 |
| | YWHAG |
| | ZBTB33 |
| | ZBTB38 |
| | ZBTB4 |
| | ZBTB4 |
| | ZC3H6 |
| | ZCCHC14 |
| | ZCRB1 |
| | ZHX3 |
| | ZMAT3 |
| | ZMIZ1 |
| | ZMYND11 |
| | ZNF106 |
| | ZNF204P |
| | ZNF277 |
| | ZNF292 |
| | ZNF318 |
| | ZNF331 |
| | ZNF395 |
| | ZNF483 |
| | ZNF652 |
| | ZNF667-AS1 |
| | ZNF746 |
| | ZNF770 |
| | ZNF91 |
| | ZZZ3 |

1. A method of producing human gastric insulin-secreting (GINS) cells comprising:
obtaining and culturing gastric stem and progenitor cells from a gastric tissue sample of a human subject;
manipulating the gastric stem cells to cause the gastric stem and progenitor cells to express a NGN3 factor, followed by a PDX1 factor, and a MAFA factor; and
culturing the manipulated cells in a serum free medium to obtain the human GINS cells, wherein the human GINS cells are insulin-secreting and glucose-responsive.

2. The method of claim 1, wherein the factors are exogenously introduced into the gastric stem and progenitor cells.

3. The method of claim 1, wherein the factors are induced endogenously by treatment with one or more chemical compounds.

4. The method of claim 2, wherein the factors are exogenously introduced into the gastric stem and progenitor cells by transduction of a viral vector, mRNA transduction, genetic engineering, or a combination thereof.

5. The method of claim 4, wherein the viral vector is a lentiviral vector or an AAV vector.

6. The method of claim 4, wherein the genetic engineering method uses CRISPR or TALEN.

7. The method according to claim 1, wherein the NGN3 factor is expressed for at least 1 day.

8. The method of claim 7, wherein the NGN3 factor is expressed for 2 days.

9. The method according to claim 1, wherein the PDX1 factor and the MAFA factor are stably expressed.

10. The method according to claim 1, wherein the expression of the NGN3 factor is transient, followed by stable expression of the PDX1 factor and the MAFA factor.

11. The method of claim 10, wherein the expression of the NGN3 factor lasts for 1-3 days, followed by stable expression of the PDX1 factor and the MAFA factor for at least 2 to 6 days.

12. The method of claim 11, wherein the expression of the NGN3 factor lasts for 2 days.

13. The method of claim 11, wherein the stable expression of the PDX1 factor and the MAFA factor last for at least 4 days.

14. A method of producing human gastric insulin-secreting (GINS) organoids comprising:
culturing the human GINS cells obtained according to claim 1 in a GINS medium for a period of time to allow aggregation of the human GINS cells into human GINS organoids, wherein the human GINS organoids are pancreatic islet-like organoids, insulin-secreting and glucose-responsive.

15. The method of claim 14, wherein the period of time is from about 6 days to about 21 days.

16. The method of claim 14, wherein the period of time is about 10 days.

17. The method of claim 14, wherein the GINS medium is a chemically defined, serum free medium.

18. The method of claim 14, wherein the GINS medium is a chemically defined, serum free medium which comprises N2, B27, and N-acetyl cysteine (“NAC”) in a basal medium.

19. The method of claim 18, wherein the basal medium is supplemented with HEPES, GlutaMAX, Primocin, NAC, B-27, N-2, Nicotinamide, A8301, and Y-27632.

20. The method of claim 19, wherein the basal medium is supplemented with 10 mM HEPES, 1× GlutaMAX, 25μ M Primocin, 500μ M NAC, 1× B-27, 1× N-2, 10 mM Nicotinamide, 1μ M A8301, and 10μ M Y-27632.

21. (canceled)

22. A population of human gastric insulin-secreting (GINS) cells, wherein the human GINS cells:
(a) are glucose-responsive and insulin-secreting,
(b) do not express certain B-cell markers such as NKX6-1 and GAD65,
(c) secrete insulin but having a granule morphology different from that of islet β-cells, and
(d) retain residual gastric gene expression.

23.-27. (canceled)

28. A preparation of human gastric insulin-secreting (GINS) organoids, wherein the human GINS organoids comprise the population of human GINS cells of claim 22.

29. A method of controlling glycemia in a human subject, comprising transplanting to the human subject (i) the population of human GINS cells according to claim 22 or (ii) a preparation of human GINS organoids comprising the population of human GINS cells according to claim 22.

30.-35. (canceled)

36. A method of treating diabetes in a human subject comprising transplanting to the human subject a mixture of the population of human GINS cells according to claim 22 and a preparation of human GINS organoids comprising the population of human GINS cells according to claim 22.
